(19) United States
(12) Patent Application Publication
(10) Pub. No.: US 2023/0221319 A1
(43) Pub. Date: Jul. 13, 2023

(54) A Method, A System, An Article, A Kit And Use Thereof For Biomolecule, Bioorganelle, Bioparticle, Cell And Microorganism Detection

(71) Applicant: National University of Singapor, Singapore (SG)

(72) Inventors: Jie Yan, Singapore (SG); Rong Li, Singapore (SG); Chen LU, Singapore (SG); Yinan Wang, Singapore (SG); Xiaodan Zhao, Singapore (SG); Yu Zhou, Singapore (SG); Shimin Le, Singapore (SG)

(21) Appl. No.: 17/928,221

(22) PCT Filed: May 31, 2021
(86) PCT No.: PCT/SG2021/050306

§ 371 Date: Nov. 28, 2022

(30) Foreign Application Priority Data
May 29, 2020 (SG) 10202005073Y

(51) Int. Cl. G01N 33/569; G01N 33/543

(52) U.S. Cl. CPC G01N 33/56983; G01N 33/54326; G01N 33/54386; G01N 2470/04; G01N 2469/10; G01N 2469/20

(57) ABSTRACT

Disclosed herein is a method of detecting the presence of a target analyte in a sample. Disclosed herein are also a system, an article, and a kit for detecting the presence of a target analyte in a sample. Disclosed herein is also the use of the system, or the article, or the kit for biomolecule, bioorganelle, bioparticle, cell and microorganism detection.

### REFERENCES TO RELATED APPLICATION

This application claims priority to Singapore application number 10202005073Y filed on 29 May 2020, the disclosure of which is hereby incorporated by reference.

### FIELD OF THE INVENTION

The present invention relates generally to the field of biomolecule, bioorganelle, bioparticle, cell and microorganism detection. In particular, the present invention relates to the use of target analyte-probe particle specific interaction for target analyte detection under a mechanical force.

### BACKGROUND ART

For virus and microorganism detection, current viral RNA or DNA based technologies are available such as RT-PCR based assays (Ian M. Mackay et al., Nucleic Acids Res, 2002, 30(6): 1292-1305). Current antibody-based detections are available, such as 1) enzyme-linked immunosorbent assay (ELISA, Eva Engvall and Peter Perlmann, J Immunol, 1972, 109 (1): 129-135), 2) immunogold labelling based assays, and 3) chemiluminescent microparticle immunoassay (Abbott Laboratories Inc.). However, these methods may require fluorescence labels or enzyme attachment which may influence results due to labelling.

Other diagnostic methods such as surface plasmon resonance (SPR) based assays depend on surface plasmonic resonance signal. The interference from surface non-specific interaction in the SPR based assays may result in false-positive signals.

There is a need to develop a detection method for biomolecule, bioparticle, bioorganelle, cell and microorganism with a different detection principle that overcome or ameliorate one or more of the limitations with the existing technologies.

### SUMMARY

A universal, fluorescence label-free method for detecting biomolecules, bioparticles, bioorganelles, cells, and microorganisms in a wide range of environmental conditions, with a single-molecule sensitivity and enhanced accuracy based on mechanical selection of specific target analyte-probe particle interaction is provided. The novel method enables fast, low-cost centralized or point-of-care diagnosis and home-based self-diagnosis of biomolecule, bioparticle, bioorganelle, cell and microorganism in suspected human samples.

The principle of the method described here also applies for fast, accurate and sensitive detection of any monovalent or multivalent targets such as viruses, antibodies, exosomes, antibodies, pathogen-associated molecules and pollutants. The method serves as a novel platform for disease diagnostics, biologics drug discovery, and environment monitoring.

In one aspect, the present disclosure refers to a method of detecting the presence of a target analyte in a sample comprising the steps of:
a) incubating the sample with a surface coated with a first sensing element, wherein the first sensing element is capable of specific binding with the target analyte, and wherein when the target analyte is present in the sample, the target analyte binds to the first sensing element present on the coated surface, concurrently with or followed by;
b) incubating the coated surface of step (a) with a plurality of probe particles, wherein the probe particles are coated with a second sensing element capable of specific interaction with the target analyte bound on the coated surface or capable of specific interaction with the first sensing element on the coated surface;
c) applying a mechanical force to separate the non-specifically bound probe particles of step (b) from the coated surface; and
d) measuring a property that reflects the amount of specifically bound probe particles on the coated surface, wherein the property is selected from the group consisting of particle density, colour density, aggregate size and combinations thereof, or a property that reflects the amount of non-specifically bound probe particles, wherein the property is selected from the group consisting of colour density, aggregate size and combinations thereof.

Advantageously, the method may be universal such that any viruses with known host receptors and/or antibodies against viral surface proteins, any antibodies with known antigens or any antigens may be detectable by the method. The method does not require the use of fluorescence labels or enzyme attachment as in ELISA for detecting a wide variety of target analytes, including microorganisms, in a wide range of environmental conditions. Hence, the method eliminates the influence from fluorescence labelling and photobleaching, and avoids the equipment cost for fluorescence detection or light absorbance detection (ELISA).

Further advantageously, single target sensitivity of detection may be achieved using specific target analyte-probe particle interaction in the method, such that low concentration of the target analyte as low as femtomolar, may be detectable, in small volume (<30 μl), thus requiring reduced sample size. These features may in turn enable early-stage diagnosis of diseases.

Still further advantageously, the method uses specific target analyte-probe particle interaction which may be selected by application of a mechanical force to ensure high accuracy and high specificity of detection. Hence, the likelihood of false positive results in the method may be significantly reduced compared to other detection methods. Parallel orthogonal detection may be achieved using a panel of probe particles or coating the surface in a multi-well plates with different first-sensing elements in different wells, to improve the throughput of detection. Further advantageously, the method does not require the use of a washing step (as is required in ELISA).

Still further advantageously, the method may be used to detect antibody with both IgG/IgM/IgA test and neutralization activity assessment. Therefore, the method may achieve both IgG/IgM/IgA test and neutralizing antibody (Nab) test in the same method.

Still further advantageously, the method generates different types of readouts allowing the method to be used as a diagnostic in different clinical settings, for instance in home-based self-diagnostics as point-of-care testing, or in a central laboratory as laboratory testing.

In another aspect, the present disclosure refers to a system for detecting the presence of a target analyte in a sample comprising:
a) a surface coated with a first sensing element, wherein the first sensing element is capable of specific binding with the target analyte;
b) a plurality of probe particles, wherein the probe particles are coated with a second sensing element capable of specific interaction with the target analyte when present or capable of specific interaction with the first sensing element on the coated surface, thereby forming specifically bound probe particles on the coated surface;
c) a mechanical force capable of separating non-specifically bound probe particles from the coated surface; and
d) a measurement means to measure a property of the specifically bound probe particles, wherein the property is selected from the group consisting of particle density, colour density, aggregate size of the specifically bound probe particles, or a property of the non-specifically bound probe particles, wherein the property is selected from the group consisting of colour density, aggregate size of the non-specifically bound probe particles.

Advantageously, the system may be specifically designed for biomolecule, bioorganelle, bioparticle, cell and microorganism detection for actual clinical trials using human samples such as serum, saliva, nasal swab and urine. Hence, system may allow detection of targeted virus in patient samples and detection of antibodies induced by specific viral infections.

In another aspect, the present disclosure refers to an article for detecting a presence of a target analyte in a sample, the article comprising:

at least one test well comprising a bottom surface coated with a first sensing element configured to bind with the target analyte in the sample, wherein the test well is configured to receive the sample and probe particles that are contained in one or more liquid mediums, the probe particles being coated with a second sensing element configured to bind with one selected from the group consisting of the first sensing element and the target analyte, such that the probe particles are specifically bound to the coated surface depending on the presence of the target analyte in the sample; and

a channel connected to the test well at a distance from the bottom surface to allow fluid communication between the channel and the test well, wherein the channel is configured to receive non-specifically bound probe particles in the test well upon an exertion of an external force on the probe particles which move the non-specifically bound probe particles away from the bottom surface.

In another aspect, the present disclosure refers to a kit for detecting a presence of a target analyte in a sample, the kit comprising:

an article as disclosed herein;

the probe particles coated with a second sensing element, the probe particles being contained in a solution; and

an instrument configured to exert the external force on the probe particles.

Advantageously, the kit may allow high-throughput screening, leading to rapid detection of a large number of samples and in a short period of time. The kit may also present a low cost and fast diagnosis of targeted analytes or therapeutic agents, which may be suitable for application in the clinical setting such as the central laboratory testing where high-volume, cheap and fast diagnosis is required.

Further advantageously, the kit may be easy to use as due to the high specificity of detection of target analyte owing to the specific target analyte-probe particle interaction and specific force required to remove non-specifically bound probe particles on coated surfaces, thus resulting in the exclusion of washing steps in the use of the kit. Hence, the use of the kit may be time efficient.

Further advantageously, the kit may be easy to use as a central laboratory test kit or a point-of-care diagnostic or a home-based self-diagnostic kit.

Advantageously, the kit may allow proper mixing of the probe particles and test samples and introduction of the mixture into the test well and removal of non-specifically bound probe particles without the aid of sophisticated laboratory equipment like micropipette. That allows the testing to be performed in a non-laboratory setting by lay individuals. Combining with smartphone-based imaging or naked human eye detection, this kit enables point-of-care testing and home-based self-testing.

In another aspect, the present disclosure refers to a method of using the kit as disclosed herein, the method comprising the steps of:

introducing the sample and the probe particles contained in the one or more liquid mediums into the article;

exerting the external force on the probe particles to move the non-specifically bound probe particles away from the bottom surface of the test well and into the channel;

analysing the probe particles in at least one selected from the group consisting of the test well and the channel, thereby determining a presence of the target analyte in the sample.

In another aspect, the present disclosure refers to use of the system, or the article, or kit as described herein for biomolecule, bioorganelle, bioparticle, cell or microorganism detection.

Advantageously, the use of the system, or the article or the kit may be specifically designed for biomolecule, bioorganelle, bioparticle, cell or microorganism detection for actual clinical trials using human samples such as serum and urine. Hence, the use of the system, or the article or the kit may allow detection of targeted viruses in patient samples or detection of antibodies induced by specific viral infections.

Further advantageously, the use of the system, or the article or the kit can be undertaken on a high-throughput scale, leading to rapid detection of a large number of samples and in a short period of time. The use of the system, or the article or the kit can also be carried out on a large-scale or be ramped up to a larger scale, if needed, easily.

### Definitions

The following words and terms used herein shall have the meaning indicated:

The term “non-specific binding” or “non-specifically bound” when used in relation to the probe particles refers to the probe particles that are not strongly binded (i.e., neither directly nor via an intervening target analyte particle) to a coated surface and therefore removed by application of a mechanical force at a faster rate.

The term “cross-linking” or “linking” when used in relation to the probe particles refers to specific binding of the probe particles, either directly or via an intervening target analyte particle, to a coated surface and therefore hardly removed by application of a mechanical force over a calibrated detection time.

The term “cross-linking assay” or “linking assay” refers to an assay where there is specific binding of the probe particles, either directly or via an intervening target analyte particle, to a coated surface and therefore hardly removed by application of a mechanical force over a calibrated time. Further, for a “cross-linking assay” or “linking assay, where there are more specifically bound probe particles on the coated surface, there is a higher level of target analyte concentration detected in the sample.

The term “blocking” or “un-linking” when used in relation to the probe particles refers to the lack of specific binding of the probe particles to a coated surface, due to the presence of target analyte in the sample which compete or prevent the binding of the probe particles to a coated surface.

The term “blocking assay” or “un-linking assay” refers to an assay where there is lack of specific binding of the probe particles to a coated surface, due to the presence of target analyte in the sample which compete or prevent the binding of the probe particles to a coated surface. Further, for a “blocking assay” or “un-linking assay, where there are more specifically bound probe particles on the coated surface, there is a lower level of target analyte concentration detected in the sample.

The term “probe particle” refers to a particle that can be perturbed (attracted or repulsed) when a mechanical force is applied to the particle, leading to its dissociation or movement.

The term “non-bait molecule” refers to a biomolecule, or a bioparticle, or a material, or a product derived from bioorganelle, virus, cell, or microorganism that when coated on a surface, is unable to crosslink target analyte particle(s) to the surface.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a method of detecting the presence of a target analyte in a sample will now be disclosed.

The method of detecting the presence of a target analyte in a sample comprising the steps of:
a) incubating the sample with a surface coated with a first sensing element, wherein the first sensing element is capable of specific binding with the target analyte, and wherein when the target analyte is present in the sample, the target analyte binds to the first sensing element present on the coated surface, concurrently with or followed by;
b) incubating the coated surface of step (a) with a plurality of probe particles, wherein the probe particles are coated with a second sensing element capable of specific interaction with the target analyte bound on the coated surface or capable of specific interaction with the first sensing element on the coated surface;
c) applying a mechanical force to separate the non-specifically bound probe particles of step (b) from the coated surface; and
d) measuring a property that reflects the amount of specifically bound probe particles on the coated surface, wherein the property is selected from the group consisting of particle density, colour density, aggregate size and combinations thereof, or a property that reflects the amount of non-specifically bound probe particles, wherein the property is selected from the group consisting of colour density, aggregate size and combinations thereof.

The method of detecting the presence of a target analyte in a sample may comprise the steps of:
a) incubating the sample with a surface coated with a first sensing element, wherein the first sensing element is capable of specific binding with the target analyte, and wherein when the target analyte is present in the sample, the target analyte binds to the first sensing element present on the coated surface, concurrently with or followed by;
b) incubating the coated surface of step (a) with a plurality of probe particles, wherein the probe particles are coated with a second sensing element capable of specific interaction with the target analyte bound on the coated surface or capable of specific interaction with the first sensing element on the coated surface;
c) applying a mechanical force to separate the non-specifically bound probe particles of step (c) from the coated surface; and
d) measuring a property that reflects the amount of specifically bound probe particles on the coated surface, wherein the property is selected from the group consisting of particle density, colour density, aggregate size and combinations thereof.

The method of detecting the presence of a target analyte in a sample may comprise the steps of:
a) incubating the sample with a surface coated with a first sensing element, wherein the first sensing element is capable of specific binding with the target analyte, and wherein when the target analyte is present in the sample, the target analyte binds to the first sensing element present on the coated surface, concurrently with or followed by;
b) incubating the coated surface of step (a) with a plurality of probe particles, wherein the probe particles are coated with a second sensing element capable of specific interaction with the target analyte bound on the coated surface or capable of specific interaction with the first sensing element on the coated surface;
c) applying a mechanical force to separate the non-specifically bound probe particles of step (b) from the coated surface onto a second surface and condensing the non-specifically bound probe particles into an aggregate on the second surface under application of the mechanical force to the second surface; and
e) measuring a property that reflects the amount of non-specifically bound probe particles, wherein the property is selected from the group consisting of colour density, aggregate size and combinations thereof.

It is possible to measure both specifically bound probe particles and non-specifically bound probe particles by following the steps as disclosed above. The measurement of the non-specifically bound probe particles may anti-correlate with the measurement of the specifically bound probe particles.

The sample may be a liquid sample. The sample may be derived or obtained from human, animal, bioorganelle, virus, cell, microorganism, or environment. The sample may comprise blood, urine, saliva, sputum, serum, liquid derived from cell or tissue.

The sample may be diluted or serially diluted. The sample may be filtered to remove contaminants. The sample may undergo pre-treatment to remove, or reduce, or destroy molecules that would compete with the target analyte in binding on the coated surface, or compete with the target analyte in binding to the probe particles, or compete with the target analyte in binding to both the coated surface and probe particles. The pre-treatment may involve a processing step selected from the group comprising of homogenization, lyzing, extraction, vortexing, agitation, dilution, heating, pressuring, centrifugation, bioseparation, dialysis, chromatography, fractionation, purification, isolation, polishing, recovery, concentration and combinations thereof. The pre-treatment may involve addition of one or more liquid mediums to the sample. The one or more liquid mediums may allow the release of the target analyte from a component of the sample. For instance, the one or more liquid mediums may lyse the virus, or microorganism, or cell, and allow the release of the target analyte from the virus, or microorganism, or intracellular component of the cell. In addition, the one or more liquid mediums may homogenize the properties of the solution.

The surface may be a hard surface. The surface may be a non-porous surface. The surface may be selected from the group consisting of glass, borosilicate glass, quartz, polymer, and metal-coated surface. The surface may be selected from the group consisting of coverslip, plate, ELISA plate, micro-titre plate and multi-well plate. The surface may have a lower light scattering property for a particular wavelength of electromagnetic waves in the visible light region compared to the probe particles. The surface may be transparent.

The surface coated with the first sensing element can be regarded as a pre-coated surface or a surface pre-coated with the first sensing element. The first sensing element coated on the surface may be the same or different as the second sensing element coated on the probe particles.

The first sensing element and the second sending element may be independently selected from the group consisting of biomolecule, bioparticle, material and product derived from bioorganelle, virus, cell, or microorganism. The first sensing element and the second sensing element may be independently selected from the group consisting of carbohydrate, polysaccharides, lipid, protein, peptide, nucleic acid, antibody, antigen, hormone, enzyme and chemical compounds. The first and second sensing elements may be a pair of antibodies recognizing different epitopes of an antigen.

The target analyte may have multiple binding sites for binding with the first sensing element and the second sensing element. The target analyte may be soluble in an aqueous medium or solvent. The target analyte may be dispersed in a liquid medium prior to incubating with the coated surface. The liquid medium may enhance the solubility of the target analyte. The liquid medium may homogenize the properties (such as pH and salt concentration) of a sample solution containing the target analyte.

The target analyte may be selected from the group consisting of cell, virus, bacteria, archaea, fungus, protozoa, algae, deoxyribonucleic acid (DNA), ribonucleic acid (RNA), protein, antibody, antigen, cell, exosome, pathogen-associated molecule, pollutant, biomarker, target receptor, intracellular material, extracellular material, product derived from bioorganelle, virus, cell, microorganism, modified biomaterial and combinations thereof. The target analyte may be multivalent. The target analyte may be a virus. The target analyte may be COVID-19 virus. The target analyte may be SARS-COV-2 receptor binding domain protein. The target analyte may be other proteins in SARS-COV-2 such as a nucleocapsid protein. The target analyte may also be a RNA of SARS-COV-2.

The probe particles may be non-magnetic, magnetic or superparamagnetic. The probe particles may be biological or non-biological particles. The probe particles may be spherical in shape. The non-magnetic probe particles may be polymer or glass.

The magnetic or superparamagnetic probe particles may be selected from the group consisting of iron, cobalt, nickel, alloy thereof, oxide thereof and combinations thereof. The magnetic or superparamagnetic probe particles may be ferromagnetic, paramagnetic or ferrimagnetic. The probe particles may be polystyrene beads encapsulated with nanosized magnetic or superparamagnetic particles. When the probe particles are magnetic or superparamagnetic, they may be condensed into an aggregate by a magnetic force applied using a permanent magnetic or an electric magnet in the form of a magnetic needle.

The probe particles may be opaque, translucent or coloured. The probe particles may be able to scatter electromagnetic waves. The probe particles may exhibit higher light scattering property for a particular wavelength of electromagnetic waves in the visible light region compared to the coated surface and the surrounding medium and thus, may be visible as a coloured patch that may be qualitatively or quantitatively analysed.

The size of the probe particles may be in the range of about 8 nm to about 100,000 nm, about 10 nm to about 100,000 nm, about 100 nm to about 100,000 nm, about 1,000 nm to about 100,000 nm, about 10,000 nm to about 100,000 nm, about 8 nm to about 10 nm, about 8 nm to about 100 nm, about 8 nm to about 1,000 nm, about 8 nm to about 10,000 nm, about 10 nm to about 100 nm, about 10 nm to about 1,000 nm, about 10 nm to about 10,000 nm, about 100 nm to about 1,000 nm, about 100 nm to about 10,000 nm, or about 1,000 nm to about 10,000 nm.

When the size of the probe particles is in the range of about 100 nm to about 100,000 nm, the probe particles may be visible under a microscope and thus, the particle density of the probe particles may be quantitatively determined by microscope imaging at a single-particle resolution.

The mechanical force may be generated from a permanent magnet, an electric magnet, a centrifuge, an acoustics, an ultrasonic wave, a laser beam, fluid motion, fluid buoyancy, or gravity. The mechanical force may be generated from instruments such as atomic force spectroscopy, optical tweezer, magnetic tweezer, acoustic force spectroscopy, microneedle, centrifuge machine, or bio-membrane. The mechanical force may be magnetic force generated by the application of a magnetic field. The mechanical force may be in the range of about 0.01 pN to about 100 pN, about 0.1 pN to about 100 pN, about 1 pN to about 100 pN, about 10 pN to about 100 pN, about 50 pN to about 100 pN, about 75 pN to about 100 pN, about 0.01 pN to about 0.1 pN, about 0.01 pN to about 1 pN, about 0.01 pN to about 10 pN, about 0.01 pN to about 50 pN, about 0.01 pN to about 75 pN, about 0.1 pN to about 1 pN, about 0.1 pN to about 10 pN, about 0.1 pN to about 50 pN, about 0.1 pN to about 75 pN, about 1 pN to about 10 pN, about 1 pN to about 50 pN, about 1 pN to about 75 pN, about 10 pN to about 50 pN, about 10 pN to about 75 pN, or about 50 pN to about 75 pN.

Advantageously, the generated mechanical force may be used as a selection tool to distinguish non-specifically bound and specifically bound probe particles, therefore reducing the possibilities of false positive. The specific target analyte-probe particle interaction can lead to cross-linking of the probe particles to the coated surface or un-linking of the probe particles from the coated surface. When the specific target analyte-probe particle interaction results in cross-linking of the probe particles to the coated surface, the probe particles are specifically bound to the coated surface via the target analyte. The application for when the specific target analyte-probe particle interaction results in cross-linking of the probe particles to the coated surface via the target analyte may be for virus detection. When the target analyte prevents the probe particles from specific binding to the coated surface, the target analyte results in un-linking of the probe particles from the coated surface, and thus the probe particles are non-specifically bound. The application for when the target analyte results in un-linking of the probe particles from the coated surface may be for neutralizing antibody detection. As non-specific binding is generally much weaker than specific binding, probe particles bound by non-specific binding will largely dissociate upon exposure to the mechanical force over a calibrated time. By quantifying the remaining bound probe particles on the coated surface, which are mainly specifically bound probe particles, after the application of the mechanical force, the presence of target analyte may be determined accurately without the need for artificial amplification.

When the property measured is colour density, or the size of the aggregate, or both the colour density and the aggregate size formed by the specifically bound probe particles, the step (d) may further comprise a step (d0) of condensing the specifically bound probe particles on the coated surface into an aggregate before step (d) by applying a magnetic force on the other side of the coated surface. The measurement of the aggregate formed by the specifically bound probe particles may enhance detection signal that can be detected by human naked eyes or quantified using a smartphone camera.

The step (d0) may be performed by placing a permanent magnet or an electric magnet in the form of a magnetic needle, on the other side of the coated surface. Due to the short distance (equivalent to the thickness of the coated surface) between specifically bound probe particles on one side of the coated surface and the magnetic needle on the other side of the coated surface, the magnetic needle may generate sufficient force to attract the specifically bound probe particles together to form an aggregate. The magnetic force applied may be in the range of 0.01 pN to 100 pN.

Where the property of the non-specifically bound probe particles is measured, the step (c) further comprises providing a second surface to contact the non-specifically bound probe particles separated from the coated surface; and condensing the non-specifically bound probe particles into an aggregate on one side of the second surface by applying the mechanical force on the other side of the second surface; wherein the mechanical force is a magnetic force. This step is thus optional if only the property of the specifically bound probe particles is measured. The magnetic force applied may be in the range of 0.01 pN to 100 pN. The aggregate may be measured for colour density, or aggregate size, or colour density and aggregate size. The measurement of the aggregate formed by the non-specifically bound probe particles may enhance detection signal by eyes or smartphone camera.

The second surface may be a hard surface. The second surface may be a non-porous surface. The second surface may be selected from the group consisting of glass, borosilicate glass, quartz, polymer, and metal-coated surface. The second surface may be a coverslip, a plate, or an enclosure. The second surface may have a lower light scattering property for a particular wavelength of electromagnetic waves in the visible light region compared to the probe particles. The second surface may be transparent.

The condensation of the non-specifically bound probe particles into an aggregate on one side of the second surface may be performed by placing a permanent magnet or an electric magnet in the form of a magnetic needle, on the other side of the second surface. Due to the short distance (equivalent to the thickness of the second surface) between non-specifically bound probe particles on one side of the second surface and the magnetic needle on the other side of the second surface, the magnetic needle may generate sufficient force to attract the non-specifically bound probe particles together to form an aggregate. The magnetic force applied may be in the range of 0.01 pN to 100 pN.

The aggregate formed on one side of the coated surface or on one side of the second surface may exhibit strong light scattering property for a particular wavelength of electromagnetic waves in the visible light region and thus, may be visible as a coloured patch of aggregate on one side of the coated surface or on one side of the second surface respectively. Consequently, the measurement of the colour density of the aggregate may be done by visualizing the one side of the coated surface or the one side of the second surface under a camera, or a smartphone-based camera, or naked human eyes, and quantifying via software colour analysis or analysing the intensity of the scattered light in the recorded images. The colour density of the aggregate formed on one side of the coated surface may positively or negatively correlate with the target analyte concentration in the sample when a cross-linking/linking assay or a blocking/un-linking assay is used respectively. The colour density of the aggregate formed on one side of the second surface may positively or negatively correlate with the target analyte concentration in the sample when a blocking/un-linking assay or a cross-linking/linking assay is used respectively.

The measurement of the colour density may be a qualitative analysis if the aggregate formed on one side of the coated surface or on one side of the second surface is visualized using the naked human eyes only. The measurement of the colour density may be a quantitative analysis if visualization of the aggregate formed on one side of the coated surface or on one side of the second surface is coupled with analysis of the intensity of light scattered due to the aggregate. The measurement of the colour density may lack sensitivity if the aggregate formed on one side of the coated surface or on one side of the second surface is insufficient to form a visible coloured patch. When the method is conducted such that the colour density or aggregate size is measured using naked human eyes only and/or smartphone-based camera only, the method may be suitable for application in a central laboratory or home-based self-diagnostic or point-of-care testing.

The measurement of the aggregate size of the aggregate formed on one side of the coated surface or on one side of the second surface may be done by visualizing the one side of the coated surface or the one side of the second surface under a camera, or a smartphone-based camera or naked human eyes and quantifying the covered area fraction of the aggregate. The aggregate size of the aggregate formed on one side of the coated surface may positively or negatively correlate with the target analyte concentration in the sample when a cross-linking/linking assay or a blocking/un-linking assay is used respectively. The aggregate size of the aggregate formed on one side of the second surface may positively or negatively correlate with the target analyte concentration in the sample when a blocking/un-linking assay or a cross-linking/linking assay is used respectively. The measurement of the aggregate size may be a quantitative analysis if visualization of the aggregate formed on one side of the second surface or on one side of the second surface is coupled with quantification of the covered area fraction of the aggregate. The measurement of the aggregate size of the aggregate may be more accurate compared to measurement of the colour density of the aggregate. When the method is conducted such that the aggregate size of the aggregate is measured using naked human eyes only and/or smartphone-based camera and/or microscope only, the method may be suitable for application in a central laboratory or home-based self-diagnostic or point-of-care testing.

The incubation time for incubating the coated surface with the sample for step (a) may be in the range of about 1 second to about 90 minutes, about 1 minute to about 90 minutes, about 10 minutes to about 90 minutes, about 50 minutes to about 90 minutes, about 70 minutes to about 90 minutes, about 1 second to about 1 minute, about 1 second to about 10 minutes, about 1 second to about 50 minutes, about 1 second to about 70 minutes, about 1 minute to about 10 minutes, about 1 minute to about 50 minutes, about 1 minute to about 70 minutes, about 10 minutes to about 50 minutes, about 10 minutes to about 70 minutes, or about 50 minutes to about 70 minutes.

The incubation time for incubating the probe particles with the coated surface for step (b) may be in the range of about 10 seconds to about 90 minutes, about 1 minute to about 90 minutes, about 10 minutes to about 90 minutes, about 50 minutes to about 90 minutes, about 70 minutes to about 90 minutes, about 10 seconds to about 1 minute, about 10 seconds to about 10 minutes, about 10 seconds to about 50 minutes, about 10 seconds to about 70 minutes, about 1 minute to about 10 minutes, about 1 minute to about 50 minutes, about 1 minute to about 70 minutes, about 10 minutes to about 50 minutes, about 10 minutes to about 70 minutes, or about 50 minutes to about 70 minutes.

When the property measured in step (d) is particle density of the specifically bound probe particles, the measurement may be done by visualizing the coated surface under a microscope, or a camera, or a smartphone-based camera, or naked human eyes and quantifying by counting the number of specifically bound probe particles on the coated surface manually, or via a software analysis counter, or analysing the covered area fraction, or the intensity of the recorded images. The quantification may be done by using a software analysis counter to count the number of specifically bound probe particles accumulated on the coated surface such as the bottom of a well in a micro-titre plate. This can be done by taking an image of the bottom of the micro-titre plate and processing the image using a software analysis counter.

The particle density may positively or negatively correlate with the target analyte concentration in the sample when a cross-linking/linking assay or a blocking/un-linking assay is used respectively. The measurement of the particle density of the specifically bound probe particles in step (d) may be a qualitative analysis if the specifically bound probe particles on the coated surface is visualized using the naked human eyes only. The measurement of the particle density of the specifically bound probe particles in step (d) may be a quantitative analysis if visualization of the specifically bound probe particles on the coated surface is coupled with counting of the specifically bound probe particles on the coated surface.

The specifically bound probe particles in step (d) may exhibit strong light scattering property for a particular wavelength of electromagnetic waves in the visible light region and thus, may be visible as a coloured patch of specifically bound probe particles on the coated surface. Consequently, the measurement of the colour density of the specifically bound probe particles may be done by visualizing the coated surface under a smartphone-based camera or naked human eyes and quantifying via software colour analysis or analysing the intensity of the scattered light in recorded image. The colour density may positively or negatively correlate with the target analyte concentration in the sample when a cross-linking/linking assay or a blocking/un-linking assay is used respectively.

The measurement of the colour density of the specifically bound probe particles in step (d) may be a qualitative analysis if the specifically bound probe particles on the coated surface is visualized using the naked human eyes only. The measurement of the colour density of the specifically bound probe particles in step (d) may be a quantitative analysis if visualization of the specifically bound probe particles on the coated surface is coupled with analysis of the intensity of light scattered due to the specifically bound probe particles on the coated surface. The measurement of the colour density of the specifically bound probe particles in step (d) may lack sensitivity if the number of specifically bound probe particles on the coated surface is insufficient to form a visible coloured patch.

The measurement of the property of particle density in step (d) by using a microscope may be more accurate compared to measurement of colour density or aggregate size. However, the use of the microscope may require its availability and the availability of trained personnel. Hence, when the method is conducted such that step (d) measures the property of particle density using a microscope, the method may be suitable for application in a central laboratory. Alternatively, if the measurement of particle density is by using naked human eyes only and/or smartphone-based camera only, the use of the microscope may not be required, and hence, eliminating the need for bulky or expensive instrument. Therefore, when the method is conducted such that step (d) measures the property of particle density using naked human eyes only and/or smartphone-based camera only, the method may be suitable for application in a home-based self-diagnostic or point-of-care testing.

The measurement of the property of colour density in step (d) may be more convenient compared to measurement of particle density or aggregate size. However, when the concentration of the target analyte is low, the amount of specifically bound probe particles in step (d) may be low and hence may not present a visible coloured patch on the coated surface resulting in low sensitivity. When the method is conducted such that step (d) measures the property of colour density using naked human eyes only and/or smartphone-based camera only, the method may be suitable for application in a central laboratory or home-based self-diagnostic or point-of-care testing.

The steps (a) and (b) may be carried out concurrently whereby a mixture of the sample and the plurality of probe particles is incubated with the coated surface.

Here, one or more liquid mediums may be added to the sample and the probe particles prior to mixing into a mixture. The mixture may be diluted or serially diluted prior to incubating the mixture with the coated surface. The one or more liquid mediums may enhance the solubility of the target analyte in the mixture, or homogenize the properties (e.g., pH, and salt concentration) of a sample solution containing the target analyte. The one or more liquid mediums may allow the release of the target analyte from a component of the sample. For instance, the one or more liquid mediums may lyse the virus, or microorganism, or cell, and allow the release of the target analyte from the virus, or microorganism, or intracellular component of the cell.

The incubation time for incubating the mixture with the coated surface may be in the range of about 1 minute to about 60 minutes, 10 minutes to about 60 minutes, about 20 minutes to about 60 minutes, about 30 minutes to about 60 minutes, about 40 minutes to about 60 minutes, about 1 minute to about 10 minutes, about 1 minute to about 20 minutes, about 1 minute to about 30 minutes, about 1 minute to about 40 minutes, about 10 minutes to 20 minutes, about 10 minutes to 30 minutes, about 10 minutes to about 40 minutes, about 20 minutes to about 30 minutes, about 20 minutes to about 40 minutes, or about 30 minutes to about 40 minutes.

When steps (a) and (b) are done concurrently, this may be viewed as one step, leading to a reduction in the overall incubation time and hence increasing the throughput of the method.

The overall duration for the method may be in the range of about 20 minutes to about 90 minutes, 40 minutes to about 90 minutes, about 50 minutes to about 90 minutes, about 60 minutes to about 90 minutes, about 80 minutes to about 90 minutes, about 20 minutes to about 40 minutes, about 20 minutes to about 50 minutes, about 20 minutes to about 60 minutes, about 20 minutes to about 80 minutes, about 40 minutes to about 50 minutes, about 40 minutes to about 60 minutes, about 40 minutes to about 80 minutes, about 50 minutes to about 60 minutes, about 50 minutes to about 80 minutes, or about 60 minutes to about 80 minutes.

Advantageously, the method may be universal such that any viruses with known host receptors and/or antibodies against viral surface proteins, any antibodies with known antigens or any antigens may be detectable by the method. The method does not require the use of fluorescence labels or enzyme attachment as in ELISA for detecting a wide variety of target analytes, including microorganisms, in a wide range of environmental conditions. Hence, the method eliminates the influence from fluorescence labelling and photobleaching, and avoids the equipment cost for fluorescence detection or light absorbance detection (ELISA).

Further advantageously, single target sensitivity of detection may be achieved using specific target analyte-probe particle interaction in the method, such that low concentration of the target analyte as low as femtomolar, may be detectable, in small volume (<30 μl), thus requiring reduced sample size. These features may in turn enable early-stage diagnosis of diseases.

Still further advantageously, the method uses specific target analyte-probe particle interaction which may be selected by application of a mechanical force to ensure high accuracy and high specificity of detection. Hence, the likelihood of false positive results in the method may be significantly reduced compared to other detection methods. Parallel orthogonal detection may be achieved using a panel of probe particles or coating the surface in a multi-well plates with different first-sensing elements in different wells, to improve the throughput of detection. Further advantageously, the method does not require the use of a washing step (as is required in ELISA).

Still further advantageously, the method may be used to detect antibody with both IgG/IgM/IgA test and neutralization activity assessment. Therefore, the method may achieve both IgG/IgM/IgA test and neutralizing antibody (Nab) test in the same method.

Still further advantageously, the method generates different types of readouts allowing the method to be used as a diagnostic in different clinical settings, for instance in home-based self-diagnostics as point-of-care testing, or in a central laboratory as laboratory testing.

Exemplary, non-limiting embodiments of a system will now be disclosed.

A system for detecting the presence of a target analyte in a sample comprising:
a) a surface coated with a first sensing element, wherein the first sensing element is capable of specific binding with the target analyte;
b) a plurality of probe particles, wherein the probe particles are coated with a second sensing element capable of specific interaction with the target analyte when present or capable of specific interaction with the first sensing element on the coated surface, thereby forming specifically bound probe particles on the coated surface;
c) a mechanical force capable of separating non-specifically bound probe particles from the coated surface; and
d) a measurement means to measure a property of the specifically bound probe particles, wherein the property is selected from the group consisting of particle density, colour density, aggregate size of the specifically bound probe particles, or a property of the non-specifically bound probe particles, wherein the property is selected from the group consisting of colour density, aggregate size of the non-specifically bound probe particles.

The system may further comprise a second surface for contacting the non-specifically bound probe particles. The second surface may be a hard surface. The second surface may be a non-porous surface. The second surface may be selected from the group consisting of glass, borosilicate glass, quartz, polymer, and metal-coated surface. The second surface may be a coverslip, a plate, or an enclosure. The second surface may have a lower light scattering property for a particular wavelength of electromagnetic waves in the visible light region compared to the probe particles. The second surface may be transparent.

The sample may be a liquid sample. The sample may be derived or obtained from human, animal, bioorganelle, virus, cell, microorganism, or environment. The sample may comprise blood, urine, saliva, sputum, serum, liquid derived from cell or tissue.

The sample may be diluted or serially diluted. The sample may be filtered to remove contaminants. The sample may undergo pre-treatment to remove, or reduce, or destroy molecules that would compete with the target analyte in binding on the coated surface, or compete with the target analyte in binding to the probe particles, or compete with the target analyte in binding to both the coated surface and probe particles. The pre-treatment may involve a processing step selected from the group comprising of homogenization, lyzing, extraction, vortexing, agitation, dilution, heating, pressuring, centrifugation, bioseparation, dialysis, chromatography, fractionation, purification, isolation, polishing, recovery, concentration and combinations thereof. The pre-treatment may involve addition of one or more liquid mediums to the sample. The one or more liquid mediums may allow the release of the target analyte from a component of the sample. For instance, the one or more liquid mediums may lyse the virus, or microorganism, or cell, and allow the release of the target analyte from the virus, or microorganism, or intracellular component of the cell. In addition, the one or more liquid mediums may homogenize the properties of the solution.

The surface may be a hard surface. The surface may be a non-porous surface. The surface may be selected from the group consisting of glass, borosilicate glass, quartz, polymer, and metal-coated surface. The surface may be selected from the group consisting of coverslip, plate, ELISA plate, micro-titre plate and multi-well plate. The surface may have a lower light scattering property for a particular wavelength of electromagnetic waves in the visible light region compared to the probe particles. The surface may be transparent.

The surface coated with the first sensing element can be regarded as a pre-coated surface or a surface pre-coated with the first sensing element. The first sensing element coated on the surface may be the same or different as the second sensing element coated on the probe particles.

The first sensing element and the second sensing element may be independently selected from the group consisting of biomolecule, bioparticle, material and product derived from bioorganelle, virus, cell, or microorganism. The first sensing element and the second sensing element may be independently selected from the group consisting of carbohydrate, polysaccharides, lipid, protein, peptide, nucleic acid, antibody, antigen, hormone, enzyme and chemical compounds. The first and second sensing elements may be a pair of antibodies recognizing different epitopes of an antigen.

The target analyte may have multiple binding sites for binding with the first sensing element and the second sensing element. The target analyte may be soluble in an aqueous medium or solvent. The target analyte may be dispersed in a liquid medium prior to incubating with the coated surface. The liquid medium may enhance the solubility of the target analyte. The liquid medium may homogenize the properties (such as pH and salt concentration) of a sample solution containing the target analyte.

The target analyte may be selected from the group consisting of cell, virus, bacteria, archaea, fungus, protozoa, algae, deoxyribonucleic acid (DNA), ribonucleic acid (RNA), protein, antibody, antigen, cell, exosome, pathogen-associated molecule, pollutant, biomarker, target receptor, intracellular material, extracellular material, product derived from bioorganelle, virus, cell, microorganism, modified biomaterial and combinations thereof. The target analyte may be multivalent. The target analyte may be a virus. The target analyte may be COVID-19 virus. The target analyte may be SARS-COV-2 receptor binding domain protein. The target analyte may be other proteins in SARS-COV-2 such as a nucleocapsid protein. The target analyte may also be a RNA of SARS-COV-2.

The probe particles may be non-magnetic, magnetic or superparamagnetic. The probe particles may be biological or non-biological particles. The probe particles may be spherical in shape. The non-magnetic probe particles may be polymer or glass.

The magnetic or superparamagnetic probe particles may be selected from the group consisting of iron, cobalt, nickel, alloy thereof, oxide thereof and combinations thereof. The magnetic or superparamagnetic probe particles may be ferromagnetic, paramagnetic or ferrimagnetic. The probe particles may be polystyrene beads encapsulated with nanosized magnetic or superparamagnetic particles. When the probe particles are magnetic or superparamagnetic, they may be condensed into an aggregate by a magnetic force applied using a permanent magnetic or an electric magnet in the form of a magnetic needle.

The probe particles may be opaque, translucent or coloured. The probe particles may be able to scatter electromagnetic waves. The probe particles may exhibit higher light scattering property for a particular wavelength of electromagnetic waves in the visible light region compared to the coated surface and the surrounding medium and thus, may be visible as a coloured patch that may be qualitatively or quantitatively analysed.

The size of the probe particles may be in the range of about 8 nm to about 100,000 nm, about 10 nm to about 100,000 nm, about 100 nm to about 100,000 nm, about 1,000 nm to about 100,000 nm, about 10,000 nm to about 100,000 nm, about 8 nm to about 10 nm, about 8 nm to about 100 nm, about 8 nm to about 1,000 nm, about 8 nm to about 10,000 nm, about 10 nm to about 100 nm, about 10 nm to about 1,000 nm, about 10 nm to about 10,000 nm, about 100 nm to about 1,000 nm, about 100 nm to about 10,000 nm, or about 1,000 nm to about 10,000 nm.

When the size of the probe particles is in the range of about 100 nm to about 100,000 nm, the probe particles may be visible under a microscope and thus, the particle density of the probe particles may be quantitatively determined by microscope imaging at a single-particle resolution.

The mechanical force may be generated from a permanent magnet, an electric magnet, a centrifuge, an acoustics, an ultrasonic wave, a laser beam, fluid motion, fluid buoyancy, or gravity. The mechanical force may be generated from instruments such as atomic force spectroscopy, optical tweezer, magnetic tweezer, acoustic force spectroscopy, microneedle, centrifuge machine, or bio-membrane. The mechanical force may be magnetic force generated by the application of a magnetic field.

The mechanical force may be in the range of about 0.01 pN to about 100 pN, about 0.1 pN to about 100 pN, about 1 pN to about 100 pN, about 10 pN to about 100 pN, about 50 pN to about 100 pN, about 75 pN to about 100 pN, about 0.01 pN to about 0.1 pN, about 0.01 pN to about 1 pN, about 0.01 pN to about 10 pN, about 0.01 pN to about 50 pN, about 0.01 pN to about 75 pN, about 0.1 pN to about 1 pN, about 0.1 pN to about 10 pN, about 0.1 pN to about 50 pN, about 0.1 pN to about 75 pN, about 1 pN to about 10 pN, about 1 pN to about 50 pN, about 1 pN to about 75 pN, about 10 pN to about 50 pN, about 10 pN to about 75 pN, or about 50 pN to about 75 pN.

Advantageously, the system may be specifically designed for biomolecules, bioparticles, bioorganelle, virus, cells and/or microorganism detection for actual clinical trials using human samples such as serum and urine. Hence, system may allow detection of targeted viruses in patient samples and detection of antibodies induced by specific viral infections.

Exemplary, non-limiting embodiments of an article, a kit and method of using the kit will now be disclosed.

FIG. 12 shows a diagram illustrating a perspective view of a kit 1200 for detecting a presence of a target analyte in samples in accordance with an example embodiment. The target analyte includes, but not limited to, viruses, antibodies, exosomes, antibodies, pathogen-associated molecules, and pollutants. The sample may be a liquid sample and may include one or more components derived or obtained from human, animal, microorganism, or environment.

The kit 1200 includes an article 1202, represented in FIG. 12 as a well plate 1204 and a water box 1206. The well plate 1204 may be transparent and may be made of a material selected from the group consisting of glass and polymer. The well plate 1204 may have dimensions similar to commercial multi-well plates to enable scaling. The well plate 1204 includes multiple test wells 1208. These test wells 1208 are coated with a first sensing element at least at bottom surfaces 1210 of the test wells 1208. The first sensing element is configured to bind the target analyte in the sample. Each of the test wells 1208 is configured to receive the samples and probe particles that are contained in one or more liquid mediums via openings 1212 of the test wells 1208.

The water box 1206 includes an enclosure 1214 and a plug 1216 that covers an aperture of the enclosure 1214. The enclosure 1214 is configured to receive the well plate 1204 upon the introduction of the liquid mediums containing the samples and probe particles into the test wells 1208. The plug 1216 is then inserted to cover the aperture of the enclosure 1214 to prevent leakage from the water box 1206 and minimize the possibility of environmental contamination.

The well plate 1204 includes a wall 1218 that extends outwardly from the openings 1212 of the test wells 1208. The enclosure 1214 includes four inner surfaces facing the well plate 1204 and one of these inner surfaces faces the wall 1218 and the test wells 1208. A channel is formed outside of the test wells 1208 between a space defined between the inner surface and the wall 1218. When the kit 1200 is in use, an external force is exerted on the probe particles inside the test wells 1208 which move non-specifically bound probe particles (i.e., probe particles that do not specifically bind the coated bottom surfaces 1210) away from the bottom surfaces 1210 of the test wells 1208. The channel is configured to receive these non-specifically bound probe particles from the test wells 1208 upon an exertion of the external force. The diameter of the test wells 1208 may be smaller or comparable to the height of the test wells to exert homogeneous level of mechanical force to the bound probe particles in the case of force exertion using magnet. 1212. The enclosure 1214 contains a transparent liquid 1220 to allow movement of the non-specifically bound probe particles into the channel. In an embodiment, the transparent liquid 1220 may include containing phosphate buffer saline (PBS) and bovine serum albumin (BSA).

The kit 1200 further includes the probe particles contained in a probe particles solution 1222. As shown in FIG. 12, the probe particles solution 1222 is contained in a microcentrifuge tube 1224. It should be noted that the probe particles can be freeze dried during storage. The probe particles solution 1222 is mixed with the sample prior to loading into the test well 1208. The probe particles are magnetic or superparamagnetic particles coated with a second sensing element. The second sensing element is configured to bind either the first sensing element or the target analyte, such that the probe particles are specifically bound to the coated bottom surface 1210 of the test wells 1208 depending on the presence of the target analyte in the samples. Hence, the presence of the target analyte can be determined by analysing (e.g., counting, estimating, image processing) the specifically and/or non-specifically bound probe particles.

Specifically, if the kit 1200 is used in a crosslinking assay, the second sensing element is configured to bind the target analyte. Accordingly, if the target analyte is present in the sample, the probe particles would specifically bound to the coated bottom surface 1210 via the target analyte, resulting in a larger quantity of specifically bound probe particles and a smaller quantity of non-specifically bound probe particles in the test wells 1208. On the other hand, if the target analyte is not present in the sample, the probe particles would not be specifically bound to the coated bottom surface 1210, resulting in no specifically bound probe particles and a larger quantity of non-specifically bound probe particles in the test wells 1208.

In contrast, if the kit 1200 is used in a blocking assay, the second sensing element is configured to bind the first sensing element. Accordingly, if the target analyte is present in the sample, the probe particles would be un-linked from the coated bottom surface 1210, resulting in a smaller quantity of specifically bound probe particles and a larger quantity of non-specifically bound probe particles in the test wells 1208. On the other hand, if the target analyte is not present in the sample, the probe particles would specifically bound to the coated bottom surface 1210, resulting in a larger quantity of specifically bound probe particles and a smaller quantity of non-specifically bound probe particles in the test wells 1208.

The first sensing element and the second sensing element may be independently selected from the group consisting of carbohydrate, polysaccharides, lipid, protein, peptide, nucleic acid, antibody, antigen, hormone, enzyme, and chemical compounds. It should be noted that the first sensing element coated on the bottom surfaces 1210 of the test wells 1208 may be the same or different as the second sensing element coated on the probe particles.

The kit 1200 further includes an instrument configured to exert the external force on the probe particles. The instrument is represented in FIG. 12 as a magnetic needles array 1226 including multiple thin magnetic needles 1228. These magnetic needles 1228 are configured to exert force to the bound probe particles and control a movement of the un-linked non-specifically bound probe particles into the channel in the article 1202 before the step of analysing the specifically and/or non-specifically bound probe particles to determine the presence of the target analyte.

In alternate embodiments, the well plate 1204 may include a system-control well and a blank-control well that receive the liquid medium including a sample and probe particles. The system-control well and a blank-control well are added to ensure that the kit 1200 is working properly, thus producing accurate results. Specifically, the system-control well is coated with the target analyte which can directly bind the second sensing element coated on the probe particles, thereby constantly producing a positive result regardless of whether the sample contains the target analyte. On the other hand, the blank-control well is coated with non-bait molecules that do not specifically bind with the target analyte, thereby constantly producing a negative result regardless of whether the sample contains the target analyte. In other words, if the system-control well does not show a positive result and the blank-control well does not show a negative result during an assay, the kit 1200 may not have worked properly and the result produced may not be accurate.

In alternate embodiments, the well plate 1204 may include a positive-control well and a negative-control well that receive the liquid medium including a sample and probe particles, wherein the sample is prepared to include and exclude the target analyte, respectively. The positive-control well and a negative-control well are added to ensure that the kit 1200 is working properly, thus producing accurate results. Specifically, the positive-control well receives liquid medium that contains target analyte such that the probe particles can specifically bind to the coated bottom surface 1210 via the target analyte, or the probe particles are un-linked from the coated bottom surface 1210 due to the target analyte, in a crosslinking and blocking assay respectively. On the other hand, the negative-control well receives liquid medium that does not contain target analyte such that the probe particles cannot specifically bind to the coated bottom surface 1210 due to the absence of the target analyte, or the probe particles can specifically bind to the coated bottom surface 1210 due to the absence of the target analyte, in a crosslinking and blocking assay respectively. In other words, if the positive-control well does not show a positive result and the negative-control well does not show a negative result during an assay, the kit 1200 may not have worked properly and the result produced may not be accurate.

In alternate embodiments, the external force exerted on the probe particles may be generated by means or methods other than the magnetic force from magnetic needles, including but not limited to, gravity, centrifuge, and fluid motion.

Advantageously, this kit 1200 contains multiple test wells 1208 which allow multiple tests to be conducted in parallel, thereby improving time and cost efficiencies. This kit 1200 is ideal in a central laboratory as laboratory testing kit, where cheap and high throughput testing is required.

FIG. 13A-13F show diagrams of the kit 1200 of FIG. 12, or a part thereof, when the kit 1200 is being used in a crosslinking assay.

FIG. 13A shows a side view of the well plate 1204 upon an introduction of the samples and the probe particles into the test wells 1208. Prior to the introduction step, the samples and probe particles are contained and incubated in the respective test tube 1224. The well plate 1204 is subsequently placed on a flat horizontal surface to allow the incubation of the samples and probe particles to take place inside the respective test wells 1208.

FIG. 13B shows a side view of the well plate 1204 during the incubation step inside the test wells 1208. As shown in FIG. 13A, the probe particles sink to the bottom surface 1210 in the test wells 1208, forming a visible layer 1302 adjacent the bottom surface 1210 of the test wells 1208.

FIG. 13C shows a side view of the well plate 1204 enclosed in the water box 1206. After the incubation step inside the test wells 1208 is completed, the well plate 1204 is inserted into the enclosure 1214 which is pre-filled with the transparent liquid 1220 such that the well plate 1204 is completely immersed in the transparent liquid 1220. During the insertion of the well plate 1204 into the enclosure 1214, the enclosure 1214 is slanted by about 30 degrees with respect to a horizontal plane to prevent a spill of the transparent liquid 1220. The enclosure 1214 is then sealed with the plug 1216 to prevent a leak of the transparent liquid 1220 when the water box 1206 is placed on a horizontal surface with the test wells 1208 facing up, as indicated by a first reference cue 1304 including a horizontal line and an upward arrow.

As can be seen in FIG. 13C, the enclosure 1214 includes an inner surface that faces the test well, represented here as inner surface 1306. The well plate 1204 includes a wall 1218 that extends outwardly from the opening 1212 of the test wells 1208. A space is defined between the inner surface 1306 and the wall 1218 outside of the test wells 1208 forming the channel, represented here as channel 1308. As shown in this figure, the channel 1308 is connected to the test wells 1208 at a distance from the bottom surface 1210. Specifically, the channel 1308 is connected to the test wells 1208 adjacent the openings 1212 of the test wells 1208, allowing fluid communication between the channel 1308 and the test wells 1208.

FIG. 13D shows a side view and a perspective view of the well plate 1204 enclosed in the water box 1206, with both the well plate 1204 and water box 1206 facing down. After the insertion of the well plate 1204 into the water box 1206, the water box 1206 is flipped upside down by 180 degrees so that the openings 1212 of the test wells 1208 face down, as indicated by a second reference cue 1310 including a horizontal line and a downward arrow. Next, the magnetic needles array 1226 is placed below the water box 1206 to exert magnetic forces to pull the probe particles downwards towards the openings 1212 of the test wells 1208. As a result, the non-specifically bound probe particles form aggregates 1312 in the channel 1308 outside the test wells 1208 above the tips of the magnetic needles 1228.

FIG. 13E shows a side view and a perspective view of the well plate 1204 enclosed in the water box 1206, with the aggregates 1312 being moved through the channel 1308 using the magnetic needles array 1226. Here, the water box 1206 is being slid above the magnetic needles array 1226 while facing down to relocate the aggregates 1312 of non-specifically bound probe particles out from the test wells 1208 to probe particles traps 1314. The probe particles traps 1314 are shallow indents formed on the wall 1218 between openings 1212 of test wells 1208 to prevent the non-specifically bound particles from moving back into the test wells 1208.

At this stage, the probe particles specifically bound to the coated bottom surface 1210 in the test wells 1208 can be analysed to determine a presence of the target analyte in the samples. The detection methods include:
a) Colour detection method based on the intensity of scattered light generated by specifically bound probe particle in the test wells 1208. An example of results obtained using this method is explained below with respect to FIG. 14A.
b) Microscope detection method by placing the article 1202 on a microscope stage for imaging. An example of results obtained using this method is explained below with respect to FIG. 14B.

Apart from the methods (a) and (b) described above, the size of the aggregates of probe particles in the test wells 1208 can be analysed to determine a presence of the target analyte in the samples. This method requires additional steps as explained in further detail below with respect to FIG. 13F, including flipping the kit 1204 together the water box 1206. The probe particles traps 1314 prevents the dissociated non-specifically bound particles from moving back into the test wells 1208. Alternatively, the size of aggregates of the dissociated probe particles which have been moved outside the test wells 1208 can be analysed to determine a presence of the target. In this case, flipping the kit together with the water box is not needed, and hence the probe particles traps 1314 is not necessary.

FIG. 13F shows a side view and a top view of the well plate 1204 enclosed in the water box 1206, with both the well plate 1204 and water box 1206 facing up. Upon moving the aggregates 1312 of non-specifically bound probe particles away from the test wells 1208, the water box 1206 is flipped back by 180 degrees, so that the openings 1212 of the test wells 1208 face up. Due to the presence of the probe particles traps 1314, the dissociated non-specifically bound probe particles which have fell into the probe particles trap 1314, is prevented from moving back into the wells after the water box 1206 is flipped back by 180 degrees.

The magnetic needles 1228 are then placed below the bottom surfaces 1210 of the test wells 1208 to gather the specifically bound probe particles into small aggregates 1316. The size of these aggregates 1316 is indicative of the amount of the specifically bound probe particles retained on the bottom surfaces 1210 and can be analysed to determine whether target analyte is present in the samples. An example of results obtained using the software analysis counter is explained below with respect to FIG. 14C.

It should be noted that the method based on the aggregates of probe particles can also be conducted on aggregates 1312 of the non-specifically bound probe particles in the probe particles trap 1314. Alternatively, the aggregates 1608 of the non-specifically bound probe particles can also be analysed when the test wells are facing down without the need of the additional step of flipping the microfluidic plate back by 180 degrees.

Advantageously, the relocation of the aggregates 1312 of non-specifically bound probe particles out from the test wells 1208 to probe particles traps 1314 improves the distinguishability between the specifically bounded probe particles and the non-specifically bounded probe particles 1312 during analysis.

FIG. 14A shows an image of a well plate 1204 incubated with liquid mediums containing different concentrations of target analyte. The results of FIG. 14A correspond to the results in Example 4. Here, the step as described above with respect to FIG. 13E is completed and an analysis is carried out using an image captured by a smartphone-based camera to determine the sensitivity of the kit 1200 to various concentrations of nucleocapsid antigen target analyte in the samples and under condition of different solutions.

The well plate 1204 includes three negative-control test wells 1208. The four test wells in the top section A of the well plate 1204 contain probe particles solution 1222 prepared using a homogenizing buffer as the solution mixed with the sample containing target analyte and the probe particles prepared in three different concentrations (0 pM, 1 pM, and 10 pM). The four test wells in the middle section B of the well plate 1204 contain probe particles solution 1222 prepared using mid-turbinate swab as the solution mixed with sample containing target analyte prepared in three different concentrations (0 pM, 1 pM, and 10 pM). The four test wells in the bottom section C of the well plate 1204 contain probe particles solution 1222 prepared using saliva as the solution mixed with sample containing target analyte prepared in three different concentrations (0 pM, 1 pM, and 10 pM). The negative-control wells 1208 correspond to target analyte of 0 pM.

As shown in FIG. 14A, the intensity of the scattered light in the negative-control wells 1208 is the lowest suggesting that the kit 1200 is working properly. The intensity of the scattered light in the test wells increases with increasing concentrations of the target analyte, suggesting that more probe particles had bound the first sensing element coated in the bottom surfaces 1210 of test wells 1208 as the concentration of the nucleocapsid antigen target analyte increases.

FIG. 14B shows an enlarged image of a test well 1208 visualized under a microscope. Here, the step as described above with respect to FIG. 13E is completed and an analysis is carried out using a high-resolution image of the test well 1208 recorded using 4× objective lens in a microscope. As shown in FIG. 14B, the probe particles 1404, represented as multiple dots on the image, specifically bound to the bottom surface 1210 of the test well 1208. These probe particles 1404 can be quantified using a software, thus providing a highly quantitative assessment of the target analyte in the sample.

FIG. 14C shows a graph illustrating the relation between normalized fraction of probe particles quantity and normalised probe particles aggregate size. Here, the step as described above with respect to FIG. 13F is completed and an analysis is carried out using an image of the aggregates 1316 captured by a smartphone-based camera to determine whether the target analyte is present in the samples.

The X-axis represents “normalised fraction of probe particles quantity” and Y-axis represents “normalised probe particles aggregate size”. As shown in the graph, the probe particles quantity increases with the increase in the size of the aggregates 1316. In other words, the size of the aggregates 1316 obtained from the image captured by a smartphone-based camera can be used to estimate the quantity of the probe particles retained in the test wells 1208. Thereafter, based on the quantity of the probe particles retained in the test wells 1208, a quantitative assessment of the target analyte in the samples can be conducted to determine whether the target analyte is present in the samples.

FIG. 15 shows a diagram illustrating a perspective view of a kit 1500 for detecting a presence of a target analyte in a sample in accordance with a further example embodiment. The target analyte includes, but not limited to, viruses, antibodies, exosomes, antibodies, pathogen-associated molecules, and pollutants. The sample may be a liquid sample and may include one or more components derived or obtained from human, animal, microorganism, or environment.

The kit 1500 includes an article, represented in FIG. 15 as a microfluidic plate 1502. The microfluidic plate 1502 may be transparent and may be made of a material selected from the group consisting of glass and polymer. The microfluidic plate 1502 includes two test wells, 1504. The test wells 1504 are coated with a first sensing element at least at bottom surfaces 1506 of the test wells 1504. The first sensing element is configured to bind the target analyte in the sample. Each of the test wells 1504 is configured to receive the sample and probe particles that are contained in one or more liquid mediums at openings 1508 of the test wells 1504.

The microfluidic plate 1502 further includes a system-control well 1510 and a blank-control well 1512 that receive the liquid medium including the sample and probe particles. The system-control well 1510 and blank-control wells 1512 are added to ensure that kit 1500 is working properly, thus producing accurate results. Specifically, the system-control well 1510 is coated with the target analyte which can directly bind a second sensing element coated on the probe particles, thereby constantly producing a positive result regardless of whether the sample contains the target analyte. On the other hand, the blank-control well is coated with non-bait molecules that do not specifically bind with the target analyte, thereby constantly producing a negative result regardless of whether the sample contains the target analyte. In other words, if the system-control well does not show a positive result and the blank-control well does not show a negative result during an assay, the kit 1500 may not have worked properly and the results produced may not be accurate.

The microfluidic plate 1502 further includes an inlet 1514 that allows the introduction of one or more liquid mediums containing the sample and probe particles. The microfluidic plate 1502 further includes loading channels 1516 that pass the liquid mediums from the inlet 1514 to the test wells 1504, system-control well 1510 and blank-control well 1512.

The microfluidic plate 1502 further includes removal channels 1524 connected to each of the wells. When the kit 1500 is in use, an external force is exerted on the probe particles inside the wells which move non-specifically bound probe particles (i.e., probe particles that do not specifically bind coated bottom surfaces 1506) away from the bottom surfaces 1506 of the wells. The removal channels 1524 are configured to receive these non-specifically bound probe particles from the wells upon an exertion of the external force.

The microfluidic plate 1502 further includes probe particles traps 1526 connected to the removal channels 1524. The probe particles traps 1526 are shallow indents formed towards a distal end of the removal channels 1524 to prevent the non-specifically bound particles from moving back into the wells.

The microfluidic plate 1502 further includes a waste reservoir 1528 connected to the probe particles traps 1526. An absorbent pad 1530 can be disposed within the waste reservoir 1528. The absorbent pad 1530 and waste reservoir 1528 are configured to prevent a leak of the liquid mediums from the microfluidic plate 1502. The microfluidic plate 1502 further includes a vent 1532 connected to the waste reservoir 1528. The vent 1532 provides an outlet for the compressed air within the microfluidic plate 1502 upon the introduction of the liquid mediums via the inlet 1514.

The kit 1500 further includes the probe particles contained in a probe particles solution 1534. As shown in FIG. 15, the probe particles solution 1534 is contained in a dropper bottle 1536. The probe particles are magnetic or superparamagnetic particles coated with a second sensing element. The second sensing element is configured to bind either the first sensing element or the target analyte, such that the probe particles are specifically bound to the coated bottom surface 1506 of the test wells 1504 depending on the presence of the target analyte in the sample. Hence, the presence of the target analyte can be determined by analysing (e.g., counting, estimating, image processing) the specifically and/or non-specifically bound probe particles.

Specifically, if the kit 1500 is used in a crosslinking assay, the second sensing element is configured to bind the target analyte. Accordingly, if the target analyte is present in the sample, the probe particles would specifically bound to the coated bottom surface 1506 via the target analyte, resulting in a larger quantity of specifically bound probe particles and a smaller quantity of non-specifically bound probe particles in the test wells 1504. On the other hand, if the target analyte is not present in the sample, the probe particles would be unlinked from the coated bottom surface 1506, resulting in no specifically bound probe particles and a larger quantity of non-specifically bound probe particles in the test wells 1504.

In contrast, if the kit 1500 is used in a blocking assay, the second sensing element is configured to bind the first sensing element. Accordingly, if the target analyte is present in the sample, the probe particles would be unlinked from the coated bottom surface 1506, resulting in a smaller quantity of specifically bound probe particles and a larger quantity of non-specifically bound probe particles in the test wells 1504. On the other hand, if the target analyte is not present in the sample, the probe particles would specifically bound to the coated bottom surface 1506, resulting in a larger quantity of specifically bound probe particles and a smaller quantity of non-specifically bound probe particles in the test wells 1504.

The first and second sensing elements may be selected from the group comprising of carbohydrate, polysaccharides, lipid, protein, peptide, nucleic acid, antibody, antigen, hormone, enzyme, and chemical compounds. It should be noted that the first sensing element coated on the bottom surface 1506 of the test wells 1504 may be the same or different as the second sensing element coated on the probe particles.

The dropper bottle 1536 is connected to a syringe 1538, including a barrel 1540 and a plunger 1542, configured to transfer the liquid medium between the dropper bottle 1536 and the microfluidic plate 1502. As shown in FIG. 15, the barrel 1540 contains a sample contained in a sample solution 1544 for the test. A filter 1546 is configured to be disposed in the barrel 1540 for removing particulates from the liquid mediums.

The kit 1500 further includes an instrument configured to exert the external force on the probe particles. The instrument is represented in FIG. 15 as a magnetic needles array 1548 including multiple thin magnetic needles 1550. These magnetic needles 1550 are configured to control the force exertion to the bound probe particles, and movement of the non-specifically bound probe particles into the removal channels 1524 in the microfluidic plate 1502 before the step of analysing the specifically and/or non-specifically bound probe particles to determine the presence of the target analyte.

In alternate embodiments, the external force exerted on the probe particles may be generated by means or methods other than the magnetic needles, including but not limited to, permanent magnets, electric magnets, centrifuge, acoustic method, ultrasonic method, laser beam, or gravity.

Advantageously, this kit 1500 is ideal as a home-based self-diagnostics kit or point-of-care testing (POCT) kit as the results can be obtained without the use of a bulky or expensive instrument such as a microscope. Specifically, the intensity of scattered light generated in the test wells 1504 and the intensity of aggregates formed inside the test wells 1504 or the probe particles trap 1526 using the magnetic needles 1550 can be easily determined by naked human eyes or analysed based on images captured by a smartphone-based camera.

FIG. 16A-16F show diagrams of the kit 1500 of FIG. 15, or a part thereof, when the kit 1500 is being used in a crosslinking assay.

FIG. 16A shows three images illustrating the steps involved in the preparation of the liquid mediums including the sample and probe particles. In the first image, the syringe 1538 pushes the sample solution 1544 through the filter 1546 into the dropper bottle 1536, where the sample solution 1544 is mixed with the probe particles solution 1534. In the second image, the syringe 1538 and dropper bottle 1536 are flipped upward to remove the air in the dropper bottle 1536. In the third image, the liquid medium containing the probe particles solution 1534 and the sample solution 1544 is ready and the dropper bottle 1536 can be connected to the inlet 1514 of the microfluidic plate 1502 together with the syringe 1538.

FIG. 16B shows a side view of the microfluidic plate 1502 upon an introduction of the liquid medium into the test well 1504. The microfluidic plate 1502 is placed on a flat horizontal surface to allow the incubation of the sample and probe particles to take place inside the test well 1504. A first reference cue 1602 including a horizontal line and an upward arrow is shown in FIG. 16B to indicate that the microfluidic plate 1502 faces up.

FIG. 16C shows a side view of the microfluidic plate 1502 during the incubation step inside the test well 1504. As shown in FIG. 16C, the probe particles sink to the bottom surface 1506 in the test well 1504, forming a visible layer 1604 adjacent the bottom surface 1506 of the test well 1504.

FIG. 16D shows a side view of the microfluidic plate 1502 facing down. After the incubation step inside the test wells 1504 is completed, the microfluidic plate 1502 is flipped upside down by 180 degrees so that the openings 1508 of the test wells 1504 face down, as indicated by a second reference cue 1606 including a horizontal line and a downward arrow. Next, the magnetic needles array 1548 is placed below the microfluidic plate 1502 to exert magnetic forces to the probe particles. The dissociated non-specifically bound probe particles are pulled downwards towards the openings 1508 of the test wells 1504. As a result, the non-specifically bound probe particles form aggregates 1608 above the tips of the magnetic needles, which are then relocated outside the test wells via the removal channel 1524 by sliding the microfluidic plate relative to the magnetic needles 1550.

FIG. 16E shows a side view of the microfluidic plate 1502 facing down, with the aggregates 1608 being moved through the removal channel 1524 using the magnetic needles array 1548. Here, the microfluidic plate 1502 is being slid above the magnetic needles array 1548 while facing down to relocate the aggregates 1608 of non-specifically bound probe particles out from the test wells 1504 to probe particles traps 1526.

At this stage, the specifically bound probe particles bound to the coated bottom surface 1506 in the test wells 1504 can be analysed to determine a presence of the target analyte in the sample. The detection methods include:
a) Colour detection method based on the intensity of scattered light generated by specifically bound probe particle in the test wells 1504.
b) Microscope detection method by placing the microfluidic plate 1502 on a microscope stage for imaging.

Apart from the methods (a) and (b) described above, the size of the aggregates of probe particles in the test wells 1504 or probe particles trap 1526 can be analysed to determine a presence of the target analyte in the sample. This method requires additional steps, as explained in further detail below with respect to FIG. 16F.

FIG. 16F shows a side view of the microfluidic plate 1502 facing up for magnetic aggregation step to be conducted in the test wells 1504. Upon moving the aggregates 1608 of non-specifically bound probe particles away from the test wells 1504, the microfluidic plate 1502 is flipped back by 180 degrees, so that the openings 1508 of the test wells 1504 face up. As a result, the non-specifically probe particles fall into the probe particles trap 1526, preventing them from moving back to the test wells.

The magnetic needles 1550 are then placed below the bottom surface 1506 of the test wells 1504 to gather the specifically bound probe particles into small aggregates 1610. The size of these aggregates 1610 is indicative of the amount of the specifically bound probe particles retained on the bottom surface 1506 and can be analysed to determine whether target analyte is present in the sample.

It should be noted that the method based on the aggregates of probe particles can also be conducted on aggregates 1608 of the non-specifically bound probe particles in the probe particles trap 1526. Alternatively, the aggregates 1608 of the non-specifically bound probe particles can also be analysed when the test wells are facing down without the need of the additional step of flipping the microfluidic plate back by 180 degrees.

Advantageously, the relocation of the aggregates 1608 of non-specifically bound probe particles out from the test wells 1504 to probe particles traps 1526 improves the distinguishability between the specifically bounded probe particles and the non-specifically bounded probe particles 1608 during analysis.

Exemplary, non-limiting embodiments of the use of a system, or an article, or a kit will now be disclosed.

The use of the system, or the article, or kit as described herein for biomolecule, bioorganelle, bioparticle, cell or microorganism detection.

The bioparticle may be a virus or a virus-like particle or an exosome. The bioparticle may be SARS-CoV-2. The biomolecule may be a protein, an antibody, an antigen, DNA, or a RNA. The microorganism may be pathological.

Advantageously, the use of the system, or the article or the kit may be specifically designed for biomolecule, bioorganelle, bioparticle, cell or microorganism detection for actual clinical trials using human samples such as serum and urine. Hence, the use of the system, or the article or the kit may allow detection of targeted viruses in patient samples or detection of antibodies induced by specific viral infections.

Further advantageously, the use of the system, or the article or the kit can be undertaken on a high-throughput scale, leading to rapid detection of a large number of samples and in a short period of time. The use of the system, or the article or the kit can also be carried out on a large-scale or be ramped up to a larger scale, if needed, easily.

### BRIEF DESCRIPTION OF THE DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1A is a schematic illustration of the general concept of the detection method of the present disclosure, where (1) denotes the sensing element, (2) denotes the target analyte, and (3) denotes the probe particle.

FIG. 1B is a schematic illustration of the detection method of the present disclosure where the specific target analyte-probe particle interaction results in cross-linking of the probe particle to the coated surface in the presence of the target analyte, leading to a positive result as shown in (a). The absence of the target analyte will lead to a negative result as shown in (b). The label (7) denotes the target analyte, (8) denotes the sensing element, and (9) denotes the probe particle.

FIG. 1C is a schematic illustration of the detection method of the present disclosure where the specific target analyte-probe particle interaction results in un-linking of the probe particle from the coated surface, leading to a positive result as shown in (a). The absence of the target analyte will lead to a negative result as shown in (b). The label (11) denotes the target analyte, (12) denotes the sensing element, and (13) denotes the probe particle.

FIG. 2 is a schematic illustration of the proof-of-concept experimental procedure for detection of a mock virus target analyte. The label (15) denotes the ACE2-coated surface, (16) denotes the target analyte (RBD-coated mock virus), and (17) denotes the superparamagnetic probe particle (ACE2-coated).

FIG. 3 is an image depicting the experimental results of the procedure of FIG. 2, where the specifically bound superparamagnetic probe particles are found at the bottom of the multi-well plates, for various mock virus target analyte concentrations.

FIG. 4 is the data illustrating the experimental results of the number of specifically bound superparamagnetic probe particles with respect to mock virus target analyte concentrations in the procedure described in FIG. 2, where: (a) two example analysing images of the resultant coated surface in the conditions of different mock virus target analyte concentrations, (b) ten example trials of the experiments with different mock virus target analyte concentrations, (c) an example trial of the experiment for 100 fM and 1 pM mock virus analyte concentrations in the sample, (d) two example trials to determine the level of cross-reactivity of the method.

FIG. 5 is a schematic illustration of a proof-of-concept experimental procedure for detection of an antibody target analyte (CR3022, an antibody to SARS-CoV-2).

FIG. 6 is a set of images depicting the experimental results of the procedure of FIG. 5.

for various antibody analyte concentrations.

FIG. 7 is the data illustrating the experimental results of the number of specifically bound superparamagnetic probe particles with respect to antibody target analyte (CR3022, an antibody to SARS-CoV-2) concentrations in the experiment to detect antibody target analyte. Data for experimental result of control experiment with no antibody target analyte (i.e. 0 M) used is presented for comparison.

FIG. 8A is an image depicting visualization and subsequent quantification of the colour density of specifically bound probe particles on a coated surface. The label “+” indicates a well containing a positive result while the label “−” indicates a well containing a negative result.

FIG. 8B is an image depicting visualization and quantification of the particle density of specifically bound probe particles on a coated surface using a microscope.

FIG. 8C is an image depicting visualization and quantification of the aggregate size of the aggregates formed by specifically bound probe particles on a coated surface. The arrows indicate the aggregates formed as a spot on the coated surface.

FIG. 8D is an image depicting visualization and quantification of the aggregate size of the aggregates formed by non-specifically bound probe particles on a second surface (position of the aggregates indicated by the white arrows). The black arrows indicate the position of the specifically bound probe particles on a coated surface.

FIG. 9A is the data illustrating the clinical validation of the method in anti-SARS-CoV-2 receptor binding domain (anti-SARS-CoV-2 RBD) antibody detection, with quantification results of antibody levels detected in the plasma samples of COVID-19 and dengue convalescent patients.

FIG. 9B is the data illustrating the clinical validation of the method in anti-SARS-CoV-2 receptor binding domain (anti-SARS-CoV-2 RBD) antibody detection, where the results obtained in the method of the present disclosure as described in FIG. 9A are directly compared against the quantification results using ELISA titer assay on the same set of plasma samples of COVID-19 and dengue convalescent patients.

FIG. 10A is an image illustrating the preliminary results of detecting the IgG antibody against SARS-CoV-2 receptor binding domain (SARS-CoV-2 RBD). The label “w/o” denotes the absence of target analyte as a negative control.

FIG. 10B is the data illustrating the corresponding quantification results of FIG. 10A.

The label “w/o” denotes the absence of target analyte as a negative control.

FIG. 11A is an image illustrating the results of specifically bound probe particles on the coated glass surface of the wells for the detection of SARS-CoV-2 nucleocapsid antigen (target analyte). The label “w/o” denotes the absence of target analyte as a negative control.

FIG. 11B is an image illustrating corresponding microscope imaging results of FIG. 11A for certain samples. The label “w/o” denotes the absence of target analyte as a negative control.

FIG. 11C is the data illustrating the corresponding quantification results of FIG. 11B. The label “w/o” denotes the absence of target analyte as a negative control.

FIG. 11D is an image illustrating the SARS-CoV-2 nucleocapsid antigen (target analyte) detection using commercially available PANBIOTM COVID-19 Ag Rapid Test Device for the indicated target analyte concentrations. The label “w/o” denotes the absence of target analyte as a negative control.

FIG. 12 shows a diagram illustrating a perspective view of a kit for detecting a presence of a target analyte in samples in accordance with an example embodiment.

FIG. 13A shows a side view of the well plate upon an introduction of the samples and the probe particles into the test wells.

FIG. 13B shows a side view of the well plate during the incubation step inside the test wells.

FIG. 13C shows a side view of the well plate enclosed in the water box.

FIG. 13D shows a side view and a perspective view of the well plate enclosed in the water box, with both the well plate and water box facing down.

FIG. 13E shows a side view and a perspective view of the well plate enclosed in the water box, with the aggregates being moved through the channel using the magnetic needles array.

FIG. 13F shows a side view and a top view of the well plate enclosed in the water box, with both the well plate and water box facing up.

FIG. 14A shows a smartphone camera image of a well plate after the non-specifically bound beads have been separated by force and relocated outside the test wells. Before the force exertion step, the wells had been incubated with liquid mediums at different concentrations of nucleocapsid proteins in different solution conditions (top four wells: working buffer; middle four wells: mid-turbinate sample; bottom four wells: saliva samples).

FIG. 14B shows an enlarged image of a test well visualized under a microscope.

FIG. 14C shows a graph illustrating the relation between normalized fraction of probe particles quantity and normalised probe particles aggregate size.

FIG. 15 shows a diagram illustrating a perspective view of a kit for detecting a presence of a target analyte in a sample in accordance with a further example embodiment.

FIG. 16A shows three images illustrating the steps involved in the preparation of the liquid mediums including the sample and probe particles.

FIG. 16B shows a side view of the microfluidic plate upon an introduction of the liquid medium into the test well.

FIG. 16C shows a side view of the microfluidic plate during the incubation step inside the test well.

FIG. 16D shows a side view of the microfluidic plate facing down.

FIG. 16E shows a side view of the microfluidic plate facing down, with the aggregates being moved through the removal channel using the magnetic needles array.

FIG. 16F shows a side view of the microfluidic plate facing up for magnetic aggregation step to be conducted in the test wells.

### DETAILED DESCRIPTION OF THE DRAWINGS

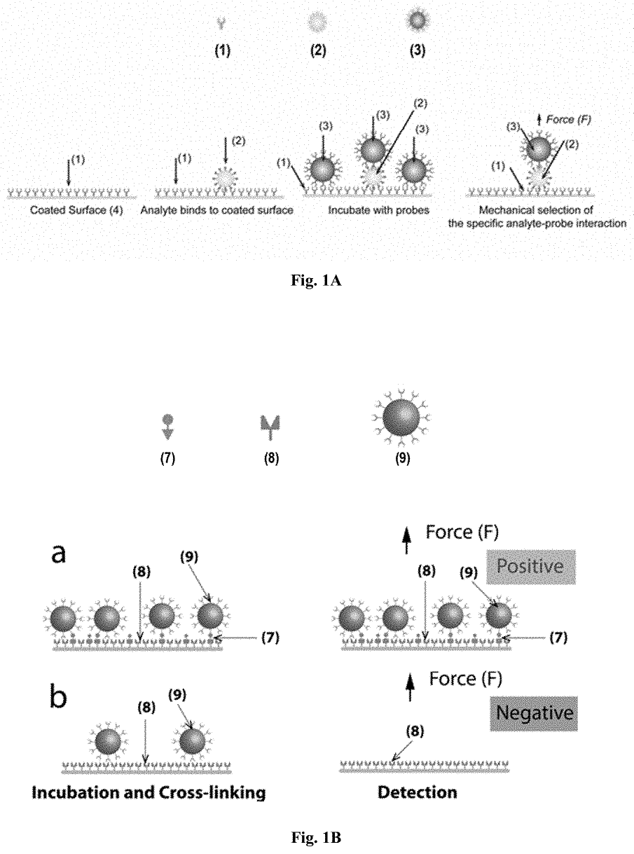

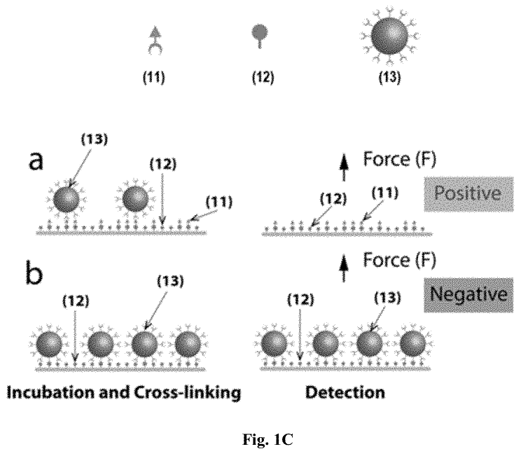

FIG. 1A is a schematic general concept of the detection method of the present disclosure. Firstly, a first sensing element (1) is pre-coated on a surface, such as a coverslip. The coated surface (4) is then incubated with a sample potentially containing the target analyte. If the target analyte (2) is present in the test sample, the target analyte (2) would bind onto the coated surface (4). Thereafter, probe particles (3) which are coated with a second sensing element are loaded onto the coated surface (4) and incubated, to allow specific interaction to occur between the target analyte (2) bound onto the coated surface and the probe particles (3). Finally, a mechanical force is applied to the probe particles (3) as a selection step, to separate the non-specifically bound probe particles from the coated surface (4). To determine the presence of target analyte (2), the number of specifically bound probe particles on the coated surface is measured. The selected specific target analyte-bound surface can be used for other downstream analysis such as sequencing.

FIG. 1B is a schematic illustration of the detection method of the present disclosure where the specific target analyte-probe particle interaction results in cross-linking of the probe particle to the coated surface in the presence of the target analyte. Firstly, a first sensing element (8) is pre-coated on a surface, such as a coverslip. The coated surface is then incubated with a sample potentially containing the target analyte. If the target analyte (7) is present in the test sample, the target analyte (7) would bind onto the coated surface. Thereafter, probe particles (9) which are coated with a second sensing element are loaded onto the surface and incubated, to allow specific interaction to occur between the target analyte (7) bound onto the coated surface and the probe particles (9). In this instance, if the target analyte (7) is present in the test sample (positive result), the probe particles (9) can be cross-linked to the coated surface via specific target analyte-probe particle interaction as shown in (a). In the absence of the target analyte (negative result) (7), the probe particles (9) will be non-specifically bound to the coated surface as shown in (b). Thereafter, a mechanical force is applied to the probe particles (9) as a selection step, to separate the non-specifically bound probe particles from the coated surface. In the absence of the targeted analyte (7) as in (b), the probe particles (9) are weakly bound the coated surface due to non-specific binding, and thus will be removed from the coated surface upon the application of the mechanical force. In contrast, for probe particles (9) that are cross-linked to coated surface via specific target analyte-probe particle interaction as in (a), they are able to withstand a certain mechanical force and remain bound to the coated surface. To determine the presence of target analyte (7), the number of specifically bound probe particles on the coated surface is measured.

FIG. 1C is a schematic illustration of the detection method of the present disclosure where the specific target analyte-probe particle interaction results in un-linking of the probe particle from the coated surface. Firstly, a first sensing element (12) is pre-coated on a surface, such as a coverslip. The coated surface is then incubated with a test sample potentially containing the target analyte (11). If the target analyte (11) is present in the test sample, the analyte (11) would bind onto the coated surface. Thereafter, probe particles (13) which are coated with a second sensing element are loaded onto the surface and incubated, to allow specific interaction to occur between the target analyte (11) bound onto the coated surface and the probe particles (13). In this instance, if the target analyte (11) is present in the sample, the target analyte (11) would prevent the cross-linking of the probe particles (13) to the coated surface via specific target analyte-probe particle interaction as shown in (a), thus the target analyte (11) results in un-linking of the probe particles (13) from the coated surface. In the absence of the target analyte (11), the probe particles (13) will be cross-linked to the coated surface as shown in (b). Thereafter, a mechanical force is applied to the probe particles (13) as a selection step, to separate the non-specifically bound probe particles from the coated surface. In the absence of the targeted analyte (11) as in (b), the probe particles (13) are strongly bound onto the coated surface due to cross-linking of the probe particles (13) with the coated surface, and thus the probe particles (13) will not be removed from the coated surface upon the application of the mechanical force. In contrast, for probe particles (13) that are un-linked from the coated surface due to the presence of the intervening target analyte (11) as in (a), the probe particles (13) are thus unable to withstand the mechanical force and will be removed from the coated surface. To determine the presence of target analyte (11), the number of remaining probe particles on the coated surface is measured.

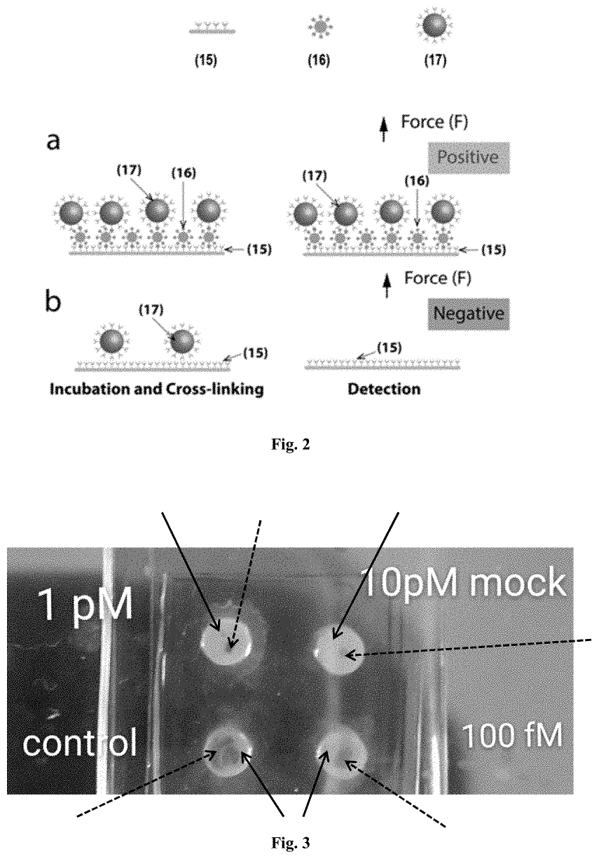

FIG. 2 is a schematic illustration of the proof-of-concept experimental procedure for detection of a mock virus target analyte, where the specific mock virus target analyte-superparamagnetic probe particle interaction results in cross-linking of the superparamagnetic probe particle to the coated surface in the presence of the mock virus target analyte, leading to a positive result as shown in (a). The absence of the mock virus target analyte will lead to a negative result as shown in (b). The label (15) denotes the ACE2-coated surface, (16) denotes the target analyte (RBD-coated mock virus), and (17) denotes the superparamagnetic probe particle (ACE2-coated).

FIG. 3 is an image depicting the experimental results of the procedure of FIG. 2, where the specifically bound superparamagnetic probe particles are found at the bottom of the multi-well plates, after applying a mechanical force in the form of a magnetic force to remove the non-specifically bound superparamagnetic probe particles in the experiment to detect mock virus target analyte, for various mock virus target analyte concentrations: 0 pM (control), 100 fM, 1 pM and 10 pM. The results can be directly visualized by the naked human eyes and distinguished by the intensity change of the surfaces, in this case, the increase in the opaque region at the bottom of the wells (shown by the full arrows (→)), due to different density of specifically bound superparamagnetic probe particles on surface with different concentrations of mock virus target analyte. The area without any superparamagnetic probe particles is shown by the dashed arrows (---->).

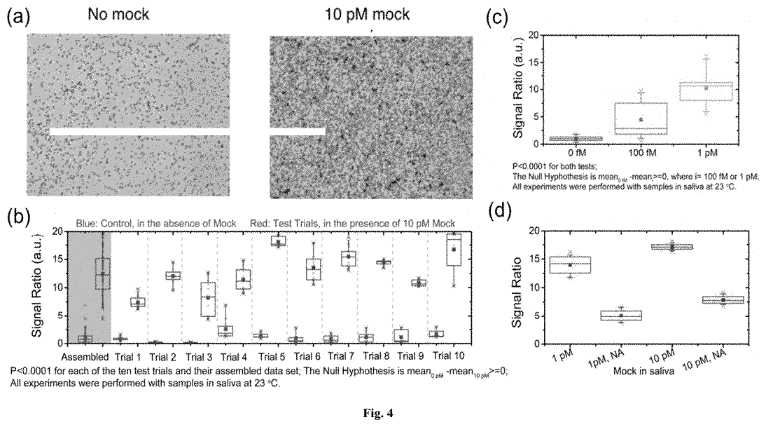

FIG. 4 is the data illustrating the experimental results of the number of specifically bound superparamagnetic probe particles with respect to mock virus target analyte concentrations in the procedure described in FIG. 2, where: (a) two example analysing images of the resultant coated surface in the conditions of 0 pM (control, left) and 10 pM (right) mock virus target analyte, (b) ten example trials of the experiments with 0 pM (control) and 10 pM mock virus analyte. For each pair of box-plot, as represented by the grey area under “Assembled” and the areas bounded by the dotted lines for “Trial 1” to “Trial 10”, the box-plot on the left (which is lower) represents the control, and the box-plot on the right (which is higher) represents the test value. All trials clearly show the detection of 10 pM mock virus target analyte in the samples, (c) an example trial of the experiment clearly detecting 100 fM and 1 pM mock virus target analyte in the sample. Similar experiments were repeated for more than 5 times, (d) two example trials to determine the level of cross-reactivity of the method. The results clearly show that the method can specifically detect the target mock virus analyte from other non-targeted analyte. “NA” denotes neutravidin coated paramagnetic beads.

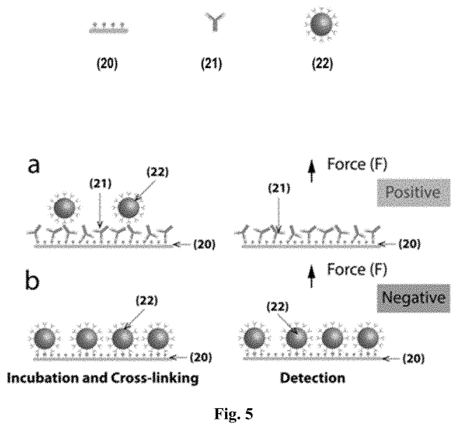

FIG. 5 is a schematic illustration of a proof-of-concept experimental procedure for detection of an antibody target analyte (CR3022, an antibody to SARS-CoV-2), where the specific antibody target analyte-superparamagnetic probe particle interaction results in un-linking of the superparamagnetic probe particle from the coated surface in the presence of the antibody target analyte, leading to a positive result as shown in (a). The absence of the antibody target analyte will lead to a negative result as shown in (b).

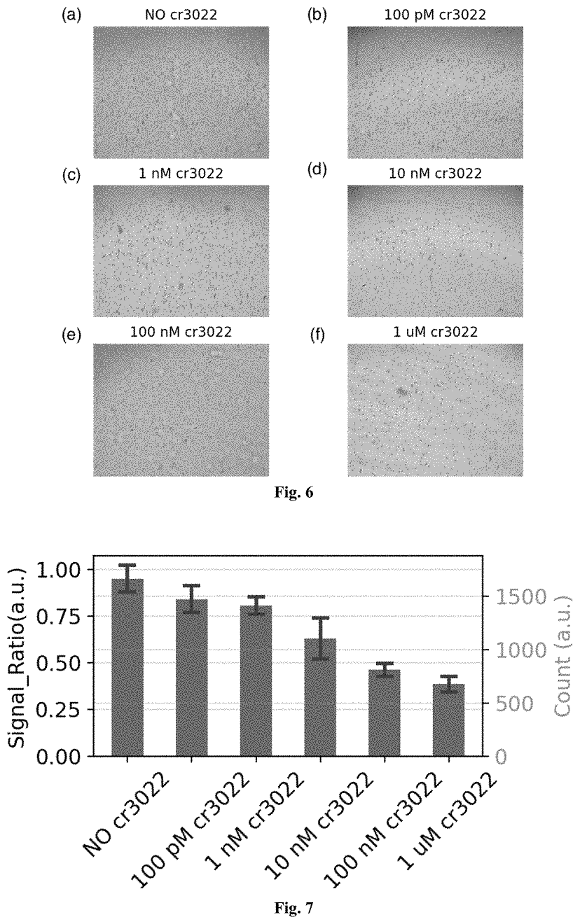

FIG. 6 is a set of images depicting the experimental results of the procedure of FIG. 5, where the specifically bound superparamagnetic probe particles after applying mechanical force in the form of magnetic force to remove non-specifically bound superparamagnetic probe particles in the experiment to detect the antibody target analyte (CR3022, an antibody to SARS-CoV-2), for various antibody target analyte concentrations: (a) 0 M, (b) 100 pM, (c) 1 nM, (d) 10 nM, (e) 100 nM and (f) 1 μM.

FIG. 7 is the data illustrating the experimental results of the number of specifically bound superparamagnetic probe particles with respect to antibody target analyte (CR3022, an antibody to SARS-CoV-2) concentrations in the experiment to detect antibody target analyte. Data for experimental result of control experiment with no antibody target analyte (i.e., 0 M) used is presented for comparison.

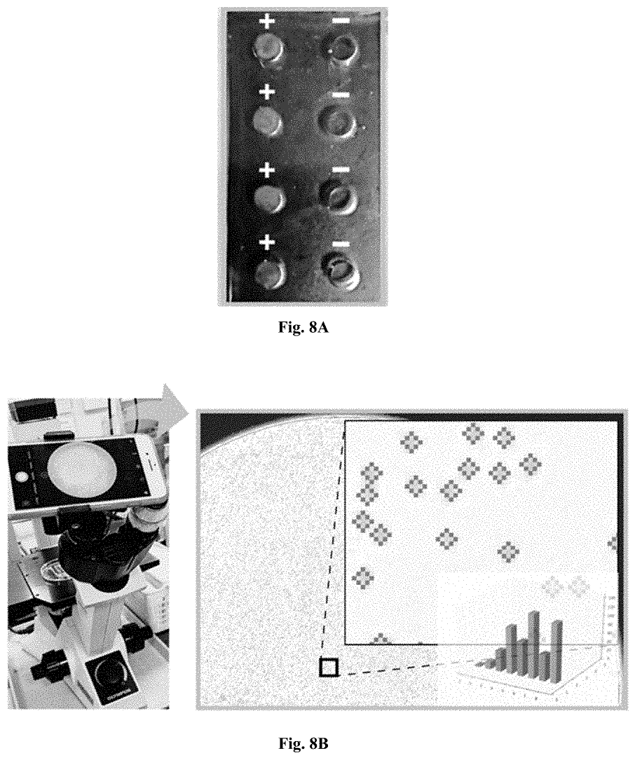

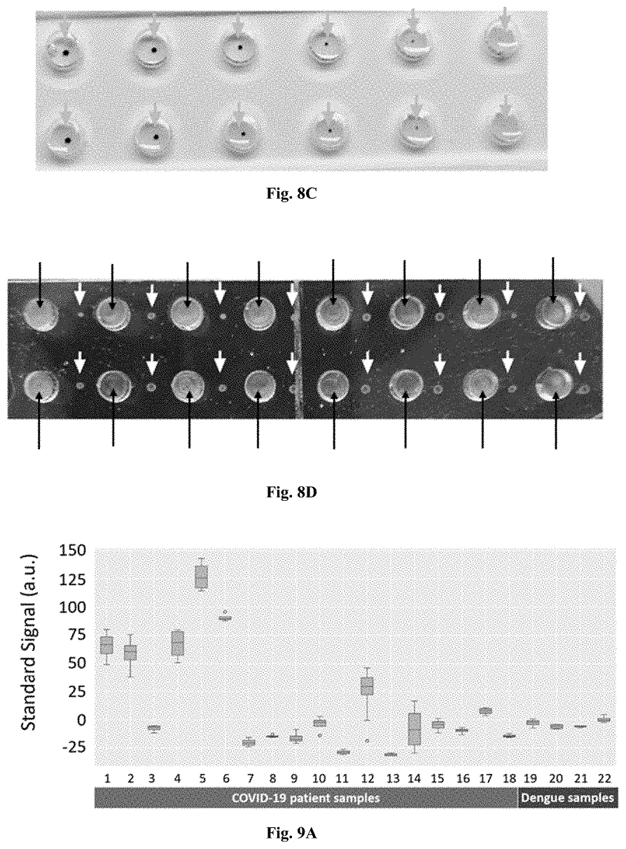

FIG. 8A is an image depicting visualization and subsequent quantification of specifically bound probe particles on a coated surface suitable in a point-of-care testing, where the coated surface has multiple-wells allowing simultaneous testing of sample(s), positive and negative controls. Since the specifically bound probe particles forms a coloured patch on the coated surface, qualitative analysis of test results by measurement of colour density by the naked human eyes is possible during visualization. The label “+” indicates a well containing a positive result while the label “−” indicates a well containing a negative result.

FIG. 8B is an image depicting visualization and quantification of specifically bound probe particles on a coated surface using a microscope. The particle density of the specifically bound probe particles per unit area may be quantified. This is a form of quantitative analysis of test results by measurement of particle density that is highly accurate and suitable for central laboratory-based testing.

FIG. 8C is an image depicting visualization and quantification of aggregates formed by specifically bound probe particles on a coated surface that is suitable in a central laboratory-based testing or point-of-care testing when used together with smartphone-based imaging, where the coated surface has multiple-wells allowing simultaneous testing of sample(s), positive and negative controls. This is a form of quantitative analysis by measurement of aggregate size or colour density of the aggregates, where the aggregate size and color density is positively or negatively correlated with the target analyte concentration in the sample when a cross-linking/linking assay or a blocking/un-linking assay is used, respectively. The arrows indicate the aggregates formed as a spot on the coated surface.

FIG. 8D is an image depicting visualization and quantification of aggregates formed by non-specifically bound probe particles on a second surface that is suitable in a central laboratory-based testing or point-of-care testing when used together with smartphone-based imaging, allowing simultaneous testing of sample(s), positive and negative controls. This is a form of quantitative analysis by measurement of aggregate size or colour density of the aggregates (white arrows denoting the aggregates), where the aggregate size and color density are positively or negatively correlated with the target analyte concentration in the sample when a blocking/un-linking assay or a cross-linking/linking assay are used, respectively. As the second surface may be in the form of a transparent enclosure enclosing the first surface, the specifically bound probe particles (black arrows denoting the specifically bound probe particles in the wells of the coated multi-well plate) may also be visible through the second surface, thus the visualization and quantification of the specifically bound probe particles may also be measured.

FIG. 10A is an image illustrating the preliminary results of detecting the IgG antibody against SARS-CoV-2 receptor binding domain (SARS-CoV-2 RBD), where (top row) images are obtained for specifically bound probe particles on coated surface at various concentrations of CR3022 antibody (target analyte), and (bottom row) images are obtained for various levels of serially diluted human serum containing CR3022 antibody (target analyte). The label “w/o” denotes the absence of target analyte as a negative control.

FIG. 10B is the data illustrating the corresponding quantification results of FIG. 10A, where the specifically bound probe particles are detected per 100×100 μm² area of coated surface in the experiment of IgG antibody against SARS-CoV-2 receptor binding domain (SARS-CoV-2 RBD), where (left) particle density data obtained for specifically bound probe particles on coated surface at various concentrations of CR3022 antibody (target analyte), and (right) particle density data obtained for various levels of serially diluted human serum containing CR3022 antibody (target analyte). The label “w/o” denotes the absence of target analyte as a negative control.

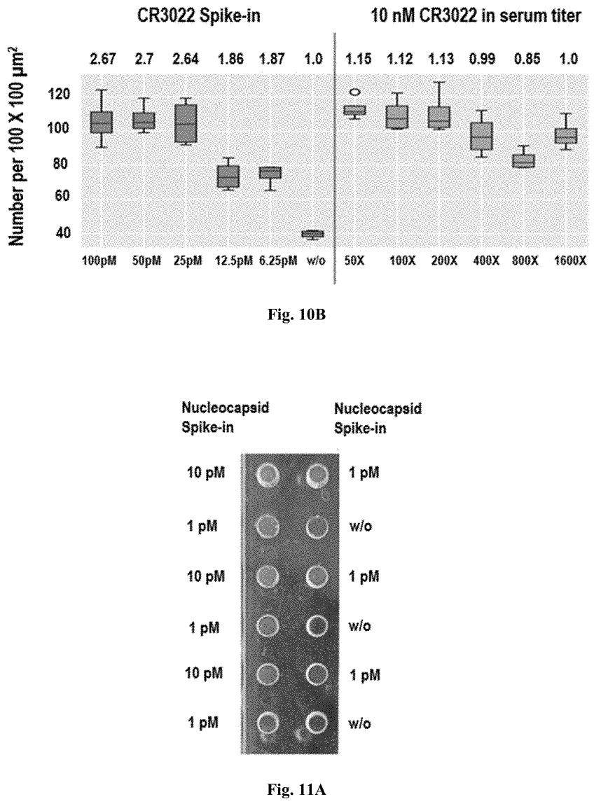

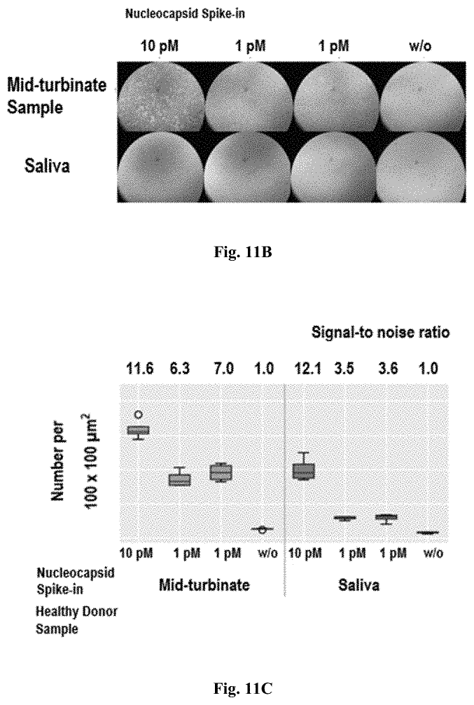

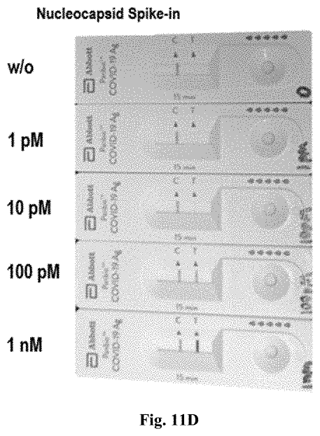

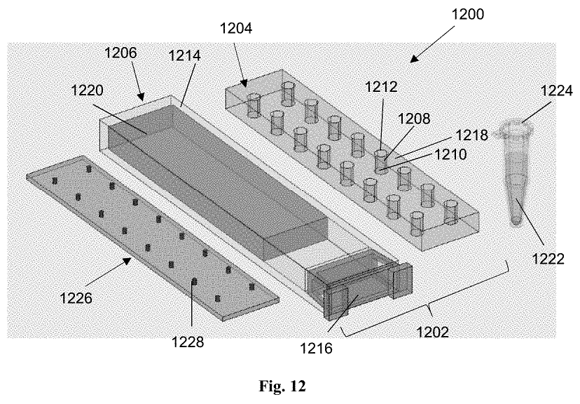

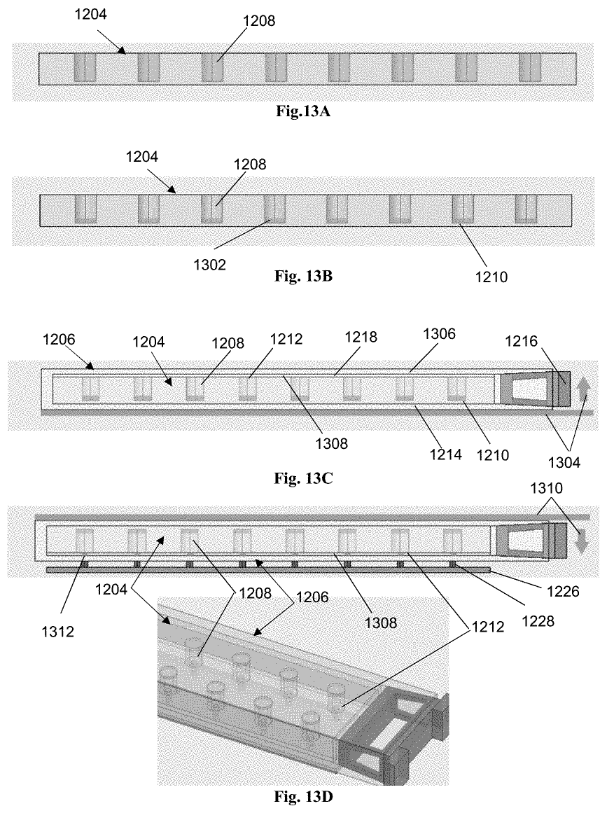

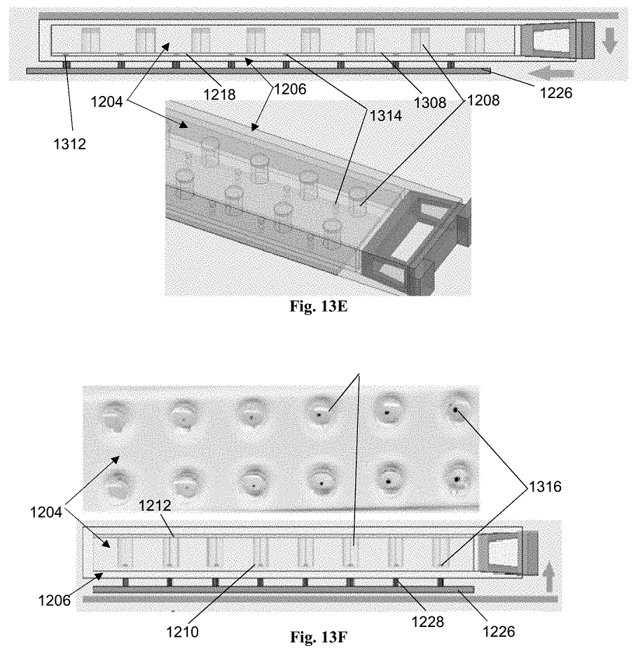

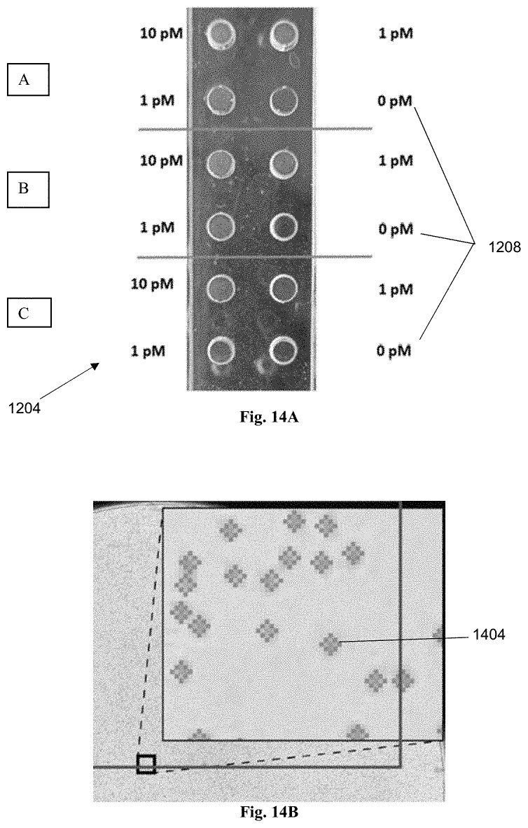

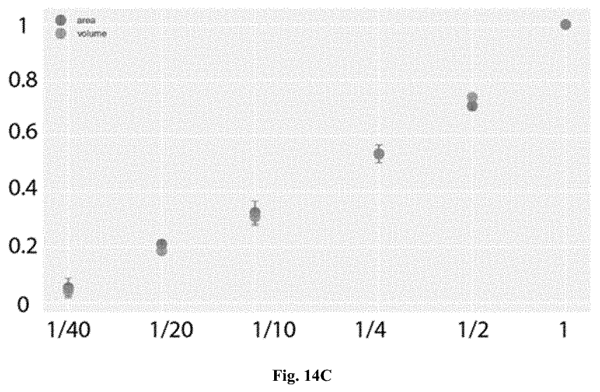

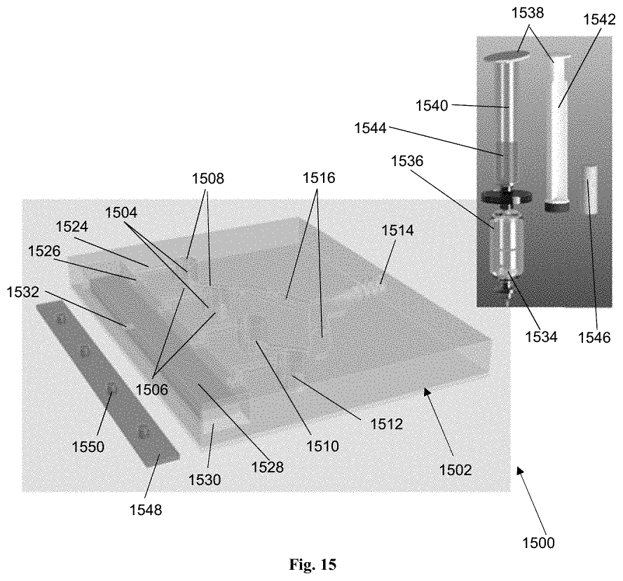

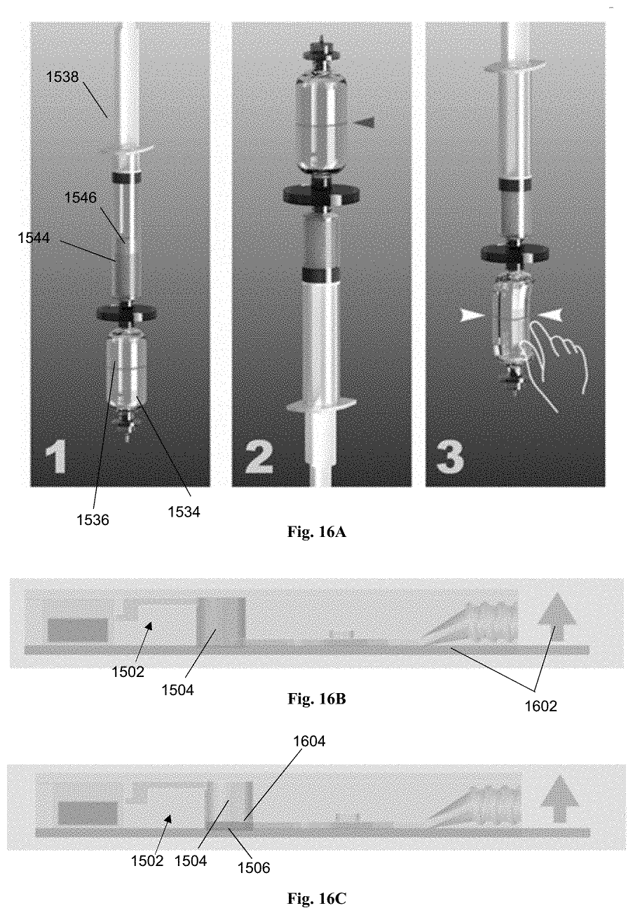

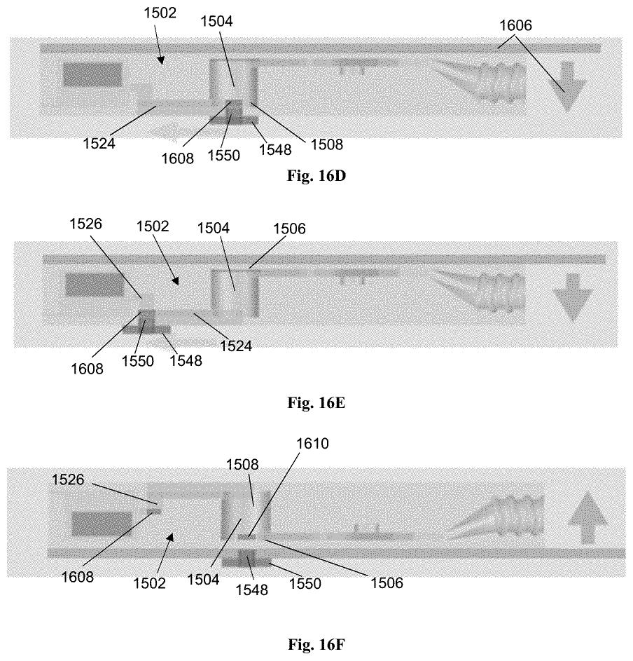

FIG. 11A is an image illustrating the results of specifically bound probe particles on the coated glass surface of the wells for the detection of SARS-CoV-2 nucleocapsid antigen (target analyte), where SARS-CoV-2 nucleocapsid antigen are spiked-in at the indicated concentrations in a homogenizing buffer (top four wells), mid-turbinate sample pre-treated with the homogenizing buffer (middle four wells), and saliva sample pre-treated the homogenizing buffer (bottom four wells). The label “w/o” denotes the absence of target analyte as a negative control.

FIG. 11B is an image illustrating corresponding microscope imaging results (using 4×magnifying lens) of specifically bound probe particles of FIG. 11A for (top row) mid-turbinate sample, and (bottom row) saliva sample in the experiment for the detection of SARS-CoV-2 nucleocapsid antigen (target analyte). The label “w/o” denotes the absence of target analyte as a negative control.

FIG. 11C is the data illustrating the corresponding quantification results of FIG. 11B, where specifically bound probe particles are detected per 100×100 μm² area of coated surface for the detection of SARS-CoV-2 nucleocapsid antigen (target analyte), where SARS-CoV-2 nucleocapsid antigen are spiked-in at the indicated concentrations in (left) mid-turbinate sample, and (right) saliva sample. The label “w/o” denotes the absence of target analyte as a negative control.

FIG. 11D is an image illustrating the SARS-CoV-2 nucleocapsid antigen (target analyte) detection using commercially available PANBIOTM COVID-19 Ag Rapid Test Device for the indicated target analyte concentrations. The label “w/o” denotes the absence of target analyte as a negative control.

### EXAMPLES

### Example 1: Detecting Mock SARS-CoV-2 Virus Analyte

To demonstrate the feasibility of detection, mock SARS-CoV-2 virus particles prepared by coating tiny spherical polystyrene particles with a layer of receptor-binding domain protein (RBD) of SARS-CoV-2 were used as target analyte (16) for this experiment as shown in FIG. 2, to mimic actual virus bioparticles. The size of the mock virus particle was ˜200 nm in diameter, which is within the range of actual virus bioparticle of 20 nm to 400 nm. The coated surface (15) used was a coverslip coated with a layer of receptor protein ACE2 as the sensing element. The superparamagnetic probe particles (17) were likewise coated with a layer of receptor protein ACE2 as the sensing element. The receptor protein ACE2 was chosen as it is known to be capable of binding with the RBD which was coated on the mock virus particles.

The coated coverslip (15) was incubated with 10 μl of liquid saliva samples containing RBD-coated mock virus particles at concentrations of 100 fM, 1 pM and 10 pM for 30 minutes at room temperature to allow the RBD-coated mock virus particles (16) to bind onto the coated coverslip (15). The volume of liquid sample used was sufficient to fully cover the surface of the coated coverslip (15). Control experiment was conducted where coated coverslip (15) was incubated with 10 μl of liquid saliva sample that does not contain the coated mock virus particles (i.e. 0 M RBD-coated mock virus particle).

Thereafter, sufficient ACE2-coated superparamagnetic probe particles (17) were loaded onto the coverslip and incubated at room temperature for 10 minutes. The coverslip was exposed to a selective mechanical force of pN-scale using superparamagnetic arrays to separate the non-specifically bound ACE2-coated superparamagnetic probe particles from the ACE2-coated coverslip. For the above-mentioned duration of the sample incubation, the level of mechanical forces applied and the duration of the applied forces, are calibrated for the mock virus target analyte and the corresponding sensing elements demonstrated by the example. These parameters may change when different sensing elements are used, or different analytes are targeted. For each system, the values should be pre-calibrated before applications.

The coated coverslip (15) that had target mock virus target analyte (16) bounded, that is the RBD-coated mock virus particle, would cross-link with the superparamagnetic probe particles (17), resulting in a much higher density of the specifically bound probe particles on the surface compared to the control, when exposed to the selective mechanical force as shown in (a) of FIG. 2.

The sample wells on the coverslips can be directly visualized by naked human eyes as shown in FIG. 3 or visualized under a microscope using 4×objective lens to record high resolution images. With the images recorded in light microscope, the remaining superparamagnetic probe particles on the coated surface can be quantified by automated counting the number of particles using proprietary written software, as shown in FIG. 4. Based on the results, the number of superparamagnetic probe particles for target analyte concentrations of 100 fM to 10 pM show significant difference from that obtained from the control. Further, the results of FIG. 4 (d) show that the method can specifically detect the mock virus target analyte from other non-targeted analyte.

The results serve as an initial proof-of-concept for the present disclosure, demonstrating the limit of detection to be possible for low concentrations of target analyte as low as sub-picomolar level. Based on this result, the method of the present disclosure may be applied in the clinical setting, to generate accurate diagnosis of virus in human sample.

### Example 2: Detecting Antibody Analyte

The capability of the present disclosure in antibody detection was also tested. In this experiment, CR3022 which is an antibody that can bind SARS-CoV-2 receptor-binding domain protein (RBD), was used as the target analyte (21) as shown in FIG. 5. The coated surface (20) used was a coverslip coated with a layer of RBD as its sensing element. The superparamagnetic probe particles (22) were coated with a layer of receptor protein ACE2 as the sensing element. The receptor protein ACE2 was chosen as it is known to be capable of binding with the RBD. Since the target antibody analyte CR3022 (21) is also known to be capable of binding with the RBD, the target analyte (21) would prevent the binding of the superparamagnetic probe particles (22) on the coated coverslip (20).

The coated coverslip (20) was incubated with 10 μl of liquid serum samples containing CR3022 antibodies at concentrations of 100 pM, 1 nM, 10 nM, 100 nM and 1 μM at room temperature to allow the CR3022 (21) to bind onto the RBD-coated coverslip for 60 minutes (20). The volume of liquid sample used was sufficient to fully cover the surface of the coated coverslip (20). Control experiment was conducted where coated coverslip (20) was incubated with 10 μl of liquid serum sample that does not contain the antibody CR3022 (i.e. 0 M CR3022).

Thereafter, the ACE2-coated superparamagnetic probe particles (22) were loaded onto the coverslip and incubated at room temperature for 10 minutes. The coverslip was exposed to a selective mechanical force of pN-scale using a magnets array to separate the non-specifically bound ACE2-coated superparamagnetic probe particles from the RBD-coated coverslip.

For the above-mentioned duration of sample incubation, the level of mechanical forces applied and the duration of the applied forces are calibrated for the specific sensing elements used in serum. These parameters may change when different sensing elements are used, or when different medium is used. For each system, the values should be pre-calibrated before applications.

The coated coverslip (20) that had target analyte CR3022 (21) bounded would prevent the cross-linking of the superparamagnetic probe particles (22) with the RBD-coated coverslip (20), hence resulting in lower amount of the superparamagnetic probe particles remaining on the coated surface after application of the mechanical force. Hence, the target analyte CR3022 (21) resulted in suppressing the stable binding of superparamagnetic probe particles (22) on the coated coverslip (20). Upon exposure to the selective mechanical force as shown in (a) of FIG. 5, the superparamagnetic probe particles (22) were removed, leading to a positive result indicated by decreased number of remained superparamagnetic probe particle on the surface. In the control experiment where the coated coverslip (20) had no target analyte (21) bounded, the superparamagnetic probe particles (22) would be stably bounded to the coated surface (20), leading to a negative result indicated by more superparamagnetic probe particles remained on the surface after being exposed to the selective mechanical force as shown in (b) of FIG. 5.

The coverslips were put under a microscope using 4×objective lens to visualise the specifically bound superparamagnetic probe particles on the coated surface as shown in FIG. 6. Based on the results, the number of specifically bound superparamagnetic probe particles on the coated surface for target analyte concentrations of 1 nM to 1 μM (FIG. 6 (c) to FIG. 6 (f)) were significantly lower than the number of specifically bound superparamagnetic probe particles on the coated surface of the control (FIG. 6 (a)).

The detailed quantification of the specifically bound superparamagnetic probe particles on the coated surface is shown in FIG. 7. The result clearly shows that the number of specifically bound superparamagnetic probe particles on the coated surface for target antibody analyte concentrations of 1 nM to 1 μM were significantly lower than the number of specifically bound superparamagnetic probe particles on the coated surface of the control, hence serving as an initial proof-of-concept for the present disclosure, demonstrating the limit of detection to be possible for low concentrations of target antibody analyte as low as nanomolar level in undiluted serum.

Based on this result, the method of the present disclosure may be applied in the clinical setting, to generate accurate diagnosis of viral antibodies in human sample.

### Example 3: Detecting Anti-SARS-CoV-2 RBD Antibody in Clinical Samples

Antibody detection: The method of the present disclosure has been applied to the detection and quantification of anti-receptor-binding domain protein (RBD) IgG antibody (target analyte) in human plasma (sample). A total of 22 convalescent plasma samples were used for the validation experiment. Of these samples, 18 plasma samples were collected from 17 patients recovered from COVID-19. One patient has donated two samples, which are samples #10 and #11, on separate dates. The sample #11 was collected 90 days after the patient's hospital admission. All other samples were collected between 29 days to 59 days post hospital admission. Besides the COVID-19 convalescent patient samples, anti-RBD antibody level in 4 plasma samples (samples #19 to #22) from patients presenting dengue fever symptoms and confirmed to be SARS-CoV-2 negative were examined using the method of the present disclosure. Although patients providing sample #20 and #22 were tested to be negative for dengue fever, these 4 samples (samples #19 to #22) serve as negative controls.

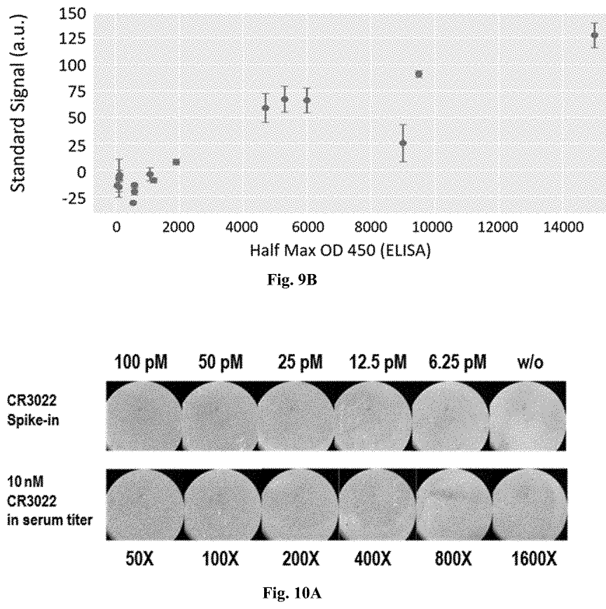

The quantification of anti-RBD antibody levels across all samples are shown in FIG. 9A. The particle density of the specifically bound probe particles on the coated surface for the samples measured are compared against commercially available SARS-CoV-2-negative human serum (Sigma Aldrich, US) of result set at 0, and against a serum with 1 μM SAD-S35 anti-RBD antibody spike-in (Acro Biosystems) of result set at 100. Based on the result shown in FIG. 9A, dengue patient samples #19 to #22 were found to be consistent, producing signals at around 0, demonstrating the absence of anti-RBD antibody in these patient samples. For COVID-19 patient samples, as seen in results of FIG. 9A for samples #1 to #18, considerably different levels of anti-RBD antibody were measured. For instance, the antibody in patient sample #5 is found to top the strength of 1 μM SAD-S35 by giving a reading of ˜127, while many others like #3, #7-11, #13 etc. give readings comparable to negative controls, suggesting the absence of such specific antibody in the patient plasma.

The antibody level in the same COVID-19 patient samples was measured using the current gold standard ELISA titer assays. The generated quantification from ELISA titer assays were represented by the half maximum absorbance at optical density at 450 nm wavelength. The data generated from the method of the present disclosure as in FIG. 9A are plotted against the results generated from the ELISA titer assays as shown in FIG. 9B. Based on the result shown in FIG. 9B, it is observed that the large the value of the tire, the higher the signal generated from the method. The method of the present disclosure when used to quantify anti-RBD antibody in human plasma is shown to be at similar accuracy to ELISA titer assays.

Quantitative titre antibody assay: The method of the present disclosure may be used to perform classic quantitative titre assay, wherein the sample is subjected to several dilutions until the signal generated falls below certain pre-set value. The method may include a standard antibody serving as a reference signal for additional level of quantification.

In this experiment, a well characterized RBD-binding IgG antibody CR3022 (Creative Biolabs) as target analyte was dissolved in a pre-diluted human serum sample (Sigma Aldrich, US) at different concentrations. A pre-diluted human serum was prepared by mixing a commercially purchased human serum and a lab-prepared homogenizing working buffer solution at a ratio of 1:49 (i.e., the human serum was diluted by 50 times). Probe particles (superparamagnetic microbeads) coated with RBD (second sensing element) were incubated with 50 μl of the pre-diluted human serum/CR3022 mixture for 15 minutes in a test tube, to allow the probe particles to link with the CR3022 target analyte in the pre-diluted human serum. Thereafter, the pre-diluted human serum was separated from the probe particles, by trapping the superparamagnetic probe particles in the test tube using a magnet and removing the pre-diluted human serum solution using a micropipette. Thereafter, the probe particles were re-suspended in 50 μl lab-prepared homogenising working buffer in a test tube, and 30 μl of the re-suspended mixture was loaded into the test well, followed by incubation for 30 minutes before analysing.

Based on the results shown in FIG. 10A, the method of the present disclosure is shown to be capable of detecting and quantifying the presence of CR3022 target analyte at concentrations as low as about 10 pM, which is about 1000 times below the dissociation constant of CR3022 target analyte to RBD sensing element. Considering that the human serum was pre-diluted by 50 times, it corresponds to about 500 pM CR3022 target analyte (equivalent to about 0.1 μg/mL CR3022 target analyte). As shown in FIG. 10B, the method of the present disclosure may be used to perform titer assay as well. A test performed using the method of the present disclosure requires approximately 45 minutes, while a typical quantitative ELISA assay requires 2 to 3 hours since it involves multiple rounds of buffer exchange. Further, a test performed using the method of the present disclosure only requires 30 μl of pre-diluted human serum solution (50 times diluted); hence only requiring a small volume of the original human serum which can be conveniently obtained by finger-prick method to extract a droplet. Although the data obtained is based on a particular RBD-binding IgG antibody (target analyte) dissolved in commercially purchased human serum, the same principle is expected to work for other RBD-binding IgG antibodies, or other antibodies against different antigens as target analyte, where a sample may be drawn from finger-prick derived whole blood sample.

### Example 4: Detecting SARS-CoV-2 Nucleocapsid Antigen in Human Saliva and Mid-Turbinate Spike-In Samples

In this experiment, a pair of antibodies recognizing different epitopes of SARS-CoV-2 nucleocapsid antigen (target analyte) was adopted as the first sensing element and second sensing element and coated on the surface and on the probe particles respectively. An homogenizing buffer was formulated to homogenize both the saliva samples and mid-turbinate samples that were pre-mixed with the antigen. Hereafter the samples mixed with the homogenizing buffer are referred to as the pre-treated sample The homogenizing buffer can also facilitate the release of the nucleocapsid antigen from the SARS-CoV-2 viral particles. The experiment was performed based on both saliva and mid-turbinate samples obtained from healthy human donors with different concentrations of nucleocapsid antigen spiked into the samples.

The results shown in FIG. 11A is derived when the experiment was conducted in a 12-well plate where the bottom glass surface was coated with the antibody belonging to the anti-SARS-CoV-2 nucleocapsid antibody pair as mentioned. Probe particles (superparamagnetic microbeads) (Invitrogen, US) were coated with the other antibody of the pair were used as the probe particles. The probe particles were mixed with homogenizing buffer, the saliva sample or the mid-turbinate sample pre-treated with the homogenizing buffer, and 30 μl of the mixture was loaded into each well. After 30 minutes of incubation, a mechanical force was exerted on the probe particles using an array of magnet to separate the non-specifically bound probe particles from the coated surface. Thereafter, the specifically bound probe particles on the coated surface were detected using a smartphone-based camera as shown in FIG. 11A, or a microscope as shown in FIG. 11B. For the microscope detection, the particle density of the specifically bound probe particles was quantified per 100×100 μm² area of the coated surface.

The results of FIG. 11A show that the specifically bound probe particles can be directly observed using a smartphone-based camera to detect the yellowish light scattered from the specifically bound probe particles, where the specifically bound probe particles is visualized as a yellow patch. The intensity of the scattered yellowish light can indicate the relative amount of the specifically bound probe particles contained in the wells. The results of FIG. 11A show that there is visually perceptible difference in the colour density for different target analyte concentrations of 10 pM, 1 pM, and 0 pM (labelled as “w/o”, negative control) in the homogenizing buffer (top 4 wells), mid-turbinate sample (middle 4 wells) pre-treated with the homogenizing buffer, or saliva sample (bottom 4 wells) pre-treated with the homogenizing buffer.

The results of FIG. 11B show the microscopic images of the coated glass surfaces of the test wells using a 4× magnifying objective lens. The specifically bound probe particles were observed as tiny, dark dots in the microscopic images, where the particle density of the specifically bound probe particles decreased as the nucleocapsid antigen (target analyte) concentration decreased. The corresponding quantification of the particle density of the specifically bound probe particles of FIG. 11B is seen in FIG. 11C, based on the particle density per 100×100 μm² area of coated surface. For both the pre-treated saliva and turbinate samples, the observed signal-to-noise ratios (the ratio of the density of the specifically bound probe particles in a sample well compared to the density of the specifically bound probe particles in the negative control well i.e., “w/o” labelled well) were more than 3, demonstrating the sensitivity of the method of the present disclosure. Similar results were obtained for the nucleocapsid antigen of a SARS-CoV-2 variant (UK variant, B1.1.7) (data not shown).

The results of FIG. 11A to 11C demonstrate that the method of the present disclosure can be used to detect the presence of ≤1 pM SARS-CoV-2 nucleocapsid antigen, when visualization and quantification is done using a smartphone-based camera or microscope for both saliva and mid-turbinate samples for ultralow viral load (i.e. target analyte concentration), as low as about 1 fM (about10⁵/mL) assuming most of the nucleocapsid antigen was released by the homogenizing buffer. This ultralow viral load achievable is comparable to the limit of detection using the current RT-qPCR detection method, where in comparison the method of the present disclosure is faster, cheaper, and easier to operate.

In another comparison shown in FIG. 11D, commercially available PANBIOTM COVID-19 Ag RAPID TEST DEVICE was observed to only detect>10 pM nucleocapsid antigen (target analyte) spiked into the working buffer of the PANBIOTM antigen test kit. Hence, the sensitivity of nucleocapsid antigen detection based on the method of the present disclosure is at least 10 times greater than that of the PANBIOTM antigen test kit. While the data presented were obtained from saliva and mid-turbinate samples, it is expected that similar detection sensitivity could be obtained for nasopharyngeal swap samples.

### INDUSTRIAL APPLICABILITY

The method, system, article and kit as disclosed herein may be used in a wide variety of diagnostic applications such as clinical testing, environmental monitoring, central laboratory testing and home-based self-diagnostic, for the of detection of the presence of a target analyte in a sample. The method, system, article and kit offer single target sensitivity of detection using specific target analyte-probe particle interaction, such that low concentrations of the target analyte, in the range of nanomolar to femtomolar, may be detectable, thus requiring reduced sample size which may in turn enable early-stage diagnosis of diseases in clinical testing.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of detecting the presence of a target analyte in a sample comprising the steps of:
a) incubating the sample with a surface coated with a first sensing element, wherein the first sensing element is capable of specific binding with the target analyte, and wherein when the target analyte is present in the sample, the target analyte binds to the first sensing element present on the coated surface, concurrently with or followed by;
b) incubating the coated surface of step (a) with a plurality of probe particles, wherein the probe particles are coated with a second sensing element capable of specific interaction with the target analyte bound on the coated surface or capable of specific interaction with the first sensing element on the coated surface;
c) applying a mechanical force to separate the non-specifically bound probe particles of step (b) from the coated surface; and
d) measuring a property that reflects the amount of specifically bound probe particles on the coated surface, wherein the property is selected from the group consisting of particle density, colour density, aggregate size and combinations thereof, or measuring a property that reflects the amount of non-specifically bound probe particles, wherein the property is selected from the group consisting of colour density, aggregate size and combinations thereof.

2. The method according to claim 1, wherein the first sensing element is the same or different as the second sensing element, and wherein the first sensing element and second sensing element are independently selected from the group consisting of biomolecule, bioparticle, material or product derived from microorganisms, or wherein the first sensing element and second sensing element are independently selected from the group consisting of carbohydrate, polysaccharides, lipid, protein, peptide, nucleic acid, antibody, antigen, hormone, enzyme, or chemical compounds.

3. The method according to claim 1, wherein the target analyte is soluble in an aqueous medium or solvent, and wherein the target analyte is dispersed in a liquid medium prior to incubating with the coated surface.

4. The method according to claim 1, wherein the probe particles are non-magnetic, magnetic or superparamagnetic particles, and wherein the size of the probe particles is in the range of 8 nm to 100,000 nm.

5. The method according to claim 1, wherein the mechanical force is generated from a permanent magnet, an electric magnet, a centrifuge, an acoustics, an ultrasonic wave, a laser beam, fluid motion, fluid buoyancy, or gravity, and wherein the mechanical force is in the range of 0.01 pN to 100 pN.

6. The method according to claim 1, wherein when the property measured is colour density, or aggregate size, or colour density and aggregate size, the step (d) further comprises a step (d0) of condensing the specifically bound probe particles on the coated surface into an aggregate before step (d) by applying a magnetic force on the other side of the coated surface.

7. The method according to claim 1, wherein the step (c) further comprises providing a second surface to contact the non-specifically bound probe particles separated from the coated surface; and
condensing the non-specifically bound probe particles into an aggregate on one side of the second surface by applying the mechanical force on the other side of the second surface;
wherein the mechanical force is a magnetic force.

8. The method according to claim 7, further comprising measuring the colour density, or aggregate size, or colour density and aggregate size of the aggregate.

9. The method according to claim 6, wherein the magnetic force is generated from a permanent magnet or an electric magnet in the form of a magnetic needle, and wherein the magnetic force is in the range of 0.01 pN to 100 pN.

10. The method according to claim 1, wherein when steps (a) and (b) are carried out concurrently, a mixture of the sample and the plurality of probe particles is incubated with the coated surface.

11. A system for detecting the presence of a target analyte in a sample comprising:
a) a surface coated with a first sensing element, wherein the first sensing element is capable of specific binding with the target analyte;
b) a plurality of probe particles, wherein the probe particles are coated with a second sensing element capable of specific interaction with the target analyte when present or capable of specific interaction with the first sensing element on the coated surface, thereby forming specifically bound probe particles on the coated surface;
c) a mechanical force capable of separating non-specifically bound probe particles from the coated surface; and
d) a measurement means to measure a property of the specifically bound probe particles, wherein the property is selected from the group consisting of particle density, colour density, aggregate size of the specifically bound probe particles, or a property of the non-specifically bound probe particles, wherein the property is selected from the group consisting of colour density, aggregate size of the non-specifically bound probe particles.

12. The system of claim 11, further comprising a second surface for contacting the non-specifically bound probe particles; and a measurement means to measure a property of the non-specifically bound probe particles, wherein the property is selected from the group consisting of colour density, aggregate size and combinations thereof.

13. An article for detecting a presence of a target analyte in a sample, the article comprising:
at least one test well comprising a bottom surface coated with a first sensing element configured to bind with the target analyte in the sample, wherein the test well is configured to receive the sample and probe particles that are contained in one or more liquid mediums, the probe particles being coated with a second sensing element configured to bind with one selected from the group consisting of the first sensing element and the target analyte, such that the probe particles are specifically bound to the coated surface depending on the presence of the target analyte in the sample; and
a channel connected to the test well at a distance from the bottom surface to allow fluid communication between the channel and the test well, wherein the channel is configured to receive non-specifically bound probe particles in the test well upon an exertion of an external force on the probe particles which move the non-specifically bound probe particles away from the bottom surface.

14. The article according to claim 13, wherein the channel:
(i) comprises a probe particles trap configured to contain the non-specifically bound probe particles received by the channel after a removal of the external force exerted on the probe particles; or
(ii) the channel is connected to the test well adjacent an opening of the test well, the opening being formed at a top portion of the test well, optionally further comprising: an enclosure including an inner surface configured to face the test well and wherein the channel comprises a space defined between the inner surface of the enclosure and a wall extends outwardly from the opening of the test well.

15. The article according to claim 13, further comprising:
a system control well including a bottom surface coated with the target analyte, wherein the system control well is configured to receive the sample and probe particles;
a blank control well including a bottom surface coated with non-bait molecules that prohibit binding with the target analyte, wherein the blank control well is configured to receive the sample and probe particles; or
a negative control well including a bottom surface coated with the first sensing element, wherein the negative control well is configured to receive the one or more liquid mediums in the absence of the target analyte.

16. (canceled)

17. (canceled)

18. (canceled)

19. (canceled)

20. A kit for detecting a presence of a target analyte in a sample, the kit comprising:
an article according to claim 13;
the probe particles coated with a second sensing element, the probe particles being contained in a solution; and
an instrument configured to exert the external force on the probe particles.

21. The kit according to claim 20, wherein the instrument comprises a magnetic needle and the probe particles comprises magnetic or superparamagnetic particles.

22. The kit according to claim 20, further comprising a syringe for introducing the sample into a dropper bottle for mixing with the solution containing the probe particles to form the one or more liquid mediums and transferring the one or more liquid mediums into the article.

23. A method of using the kit according to claim 20, the method comprising:
introducing the sample and the probe particles contained in the one or more liquid mediums into the test wells of the article;
exerting the external force on the probe particles to move the non-specifically bound probe particles away from the bottom surface of the test well and into the channel;
analysing the probe particles in at least one selected from the group consisting of the test well and the channel, thereby determining a presence of the target analyte in the sample.

24. The method of using the kit according to claim 23, wherein the step of exerting the external force on the probe particles comprises:
turning the article upside down; and
controlling a movement of the non-specifically bound probe particles into the channel using a magnetic needle.

25. (canceled)
